(19) United States
(12) Patent Application Publication
(10) Pub. No.: US 2020/0345058 A1
(43) Pub. Date: Nov. 5, 2020

(54) Nicotine Oxalic Acid Formulations

(71) Applicant: JUUL Labs, Inc., San Francisco, CA (US)

(72) Inventor: Adam Bowen, San Mateo, CA (US)

(21) Appl. No.: 16/323,914

(22) PCT Filed: Aug. 8, 2017
(86) PCT No.: PCT/US17/45996

§ 371 Date: Feb. 7, 2019

(51) Int. Cl. A24B 15/167; A24F 40/10; A61K 31/465

(52) U.S. Cl. CPC A24B 15/167; A61K 31/465; A24F 40/10

(57) ABSTRACT

A liquid nicotine formulation for generating an inhalable aerosol in an electronic cigarette comprising nicotine and oxalic acid in which there is an excess of oxalic.

### CROSS REFERENCE TO RELATED APPLICATIONS

This application claims the benefit of U.S. Provisional Application No. 62/372,261, filed Aug. 8, 2016.

### BACKGROUND

Electronic inhalable aerosol devices (e.g., vaporization devices, electronic vaping devices, and the like), particularly electronic aerosol devices, typically utilize a vaporizable material that is vaporized to create an aerosol vapor capable of delivering an active ingredient to a user for therapeutic and smoking pleasure. Such devices are preferred over combustible cigarettes for a variety of reasons, including grave health concerns for individuals that regularly smoke combustible cigarettes. Thus, there is a need in the art to provide electronic inhalable aerosol devices that provide attributes similar to combustible cigarettes, but reduce or eliminate some or all of the health concerns related to combustible cigarettes. Provided herein are solutions to these and other problems in the art.

### SUMMARY

In an aspect, a method of delivering (or administrating) protonated nicotine to a user of an electronic cigarette is provided (also referred to herein as a subject). The method includes (a) operating an electronic cigarette including a liquid nicotine formulation, the liquid nicotine formulation including nicotine, oxalic acid and a biologically acceptable liquid carrier; (b) heating the liquid nicotine formulation to an operating temperature, such that the heating provides an inhalable aerosol including an effective amount of protonated nicotine; and (c) inhaling the inhalable aerosol.

In an aspect, a method of delivering (or administrating) protonated nicotine to a user of an electronic cigarette is provided (also referred to herein as a subject). The method includes (a) heating the liquid nicotine formulation to an operating temperature using an electronic cigarette including a liquid nicotine formulation, the liquid nicotine formulation including nicotine, oxalic acid and a biologically acceptable liquid carrier, such that the heating provides an inhalable aerosol including an effective amount of protonated nicotine; and (b) inhaling the inhalable aerosol.

In another aspect, a liquid nicotine formulation includes nicotine, oxalic acid and a biologically acceptable liquid carrier is provided. Upon heating the liquid nicotine formulation, an inhalable aerosol is formed including an effective amount of protonated nicotine.

### BRIEF DESCRIPTION OF THE DRAWINGS

FIG. 1 illustrates results of heart rate data measured for six minutes from start of puffing. Y-axis is heart rate (bpm) and X-axis represent duration of the test (−60 to 180 seconds);

FIG. 2 illustrates results of heart rate data measured for ten minutes from start of puffing. Y-axis is heart rate (bpm) and X-axis represents duration of the test (0 to 10 minutes);

FIG. 3 illustrates the calculated vapor pressures of various acids relative to nicotine;

FIG. 4 illustrates the pharmacokinetic profiles for eight test articles in a blood plasma study;

FIG. 5 illustrates the comparison of Cₘₐₓ and Tₘₐₓ for eight test articles in a blood plasma study;

FIG. 6 illustrates the comparison of Cₘₐₓ and AUC for eight test articles in a blood plasma study;

FIG. 7 depicts an example embodiment of an electronic cigarette having a fluid storage compartment comprising an embodiment nicotine salt formulation described herein; and

FIG. 8 depicts an example embodiment of an electronic cigarette cartomizer having a fluid storage compartment, a heater, and comprising an embodiment nicotine salt formulation described herein.

### DETAILED DESCRIPTION

### Definitions

As used in this specification and the claims, the singular forms “a,” “an,” and “the” include plural referents unless the context clearly dictates otherwise.

The term “organic acid” as used herein, refers to an organic compound with acidic properties (e.g., by Brønsted-Lowry definition, or Lewis definition). A common organic acid is the carboxylic acids, whose acidity is associated with their carboxyl group —COOH. A dicarboxylic acid possesses two carboxylic acid groups. The relative acidity of an organic is measured by its pKₐ value and one of skill in the art knows how to determine the acidity of an organic acid based on its given pKa value. The term “keto acid” as used herein, refers to organic compounds that contain a carboxylic acid group and a ketone group. Common types of keto acids include alpha-keto acids, or 2-oxoacids, such as pyruvic acid or oxaloacetic acid, having the keto group adjacent to the carboxylic acid; beta-keto acids, or 3-oxoacids, such as acetoacetic acid, having the ketone group at the second carbon from the carboxylic acid; gamma-keto acids, or 4-oxoacids, such as levulinic acid, having the ketone group at the third carbon from the carboxylic acid.

The term “electronic cigarette” or “e-cigarette” or “low temperature vaporization device” as used herein, refers to an electronic inhaler that vaporizes a liquid solution into an aerosol mist, simulating the act of tobacco smoking. The liquid solution typically includes a formulation comprising nicotine and optionally further ingredients. There are many electronic cigarettes which do not resemble conventional cigarettes at all. The amount of nicotine contained can be chosen by the user via the inhalation. In embodiments, an electronic cigarette contains three components: a plastic cartridge that serves as a mouthpiece and a reservoir for liquid, an “atomizer” that vaporizes the liquid, and a battery. In embodiments, an electronic cigarettes may further include a combined atomizer and reservoir, called a “cartomizer” that may or may not be disposable, a mouthpiece that may be integrated with the cartomizer or not, and a battery.

The terms “deliver”, “delivering”, “administered” and “administering”, as used interchangeably herein, refer to providing an effective amount of an inhalable aerosol generated from a liquid nicotine formulation with an electronic cigarette or low temperature vaporization device in accordance with the teachings of the present disclosure. For example, the inhalable aerosol can be delivered or administered with an electronic cigarette or low temperature vaporization device in an effective amount through the mouth or nose as described herein or as would be apparent to one of skill in the art upon reading the present disclosure.

An “effective amount” is an amount sufficient to provide a level of nicotine or protonated nicotine to the user (e.g. in the blood or in the lung) to impart a nicotine-related biological effect.

The terms “oxalic acid functional group” and “oxalic acid functional groups” as used herein, refer to the two carboxyl functional groups (—COOH) found on the oxalic acid molecule. For example, a 1:1 nicotine:oxalic acid molar ratio is actually a 1:2 nicotine:oxalic acid functional group molar ratio.

The term “nicotine-related biological effect” is an effect that is detectable by the user (e.g. subject) and includes, but is not limited to, a stimulating effect or a relaxing effect. In embodiments, the nicotine-related biological effect is a stimulating effect. A stimulating effect may manifest as, for example, an increase in heart rate, an increase in blood pressure, or a feeling of satisfaction (e.g., physical satisfaction or emotional satisfaction) of a user. In embodiments, the nicotine-related biological effect is an increase in heart rate. The increase in heart rate may be achieved, for example, within about 20 seconds, about 40 seconds, about 60 seconds, about 80 seconds, about 100 seconds, about 120 seconds, about 140 seconds, about 160 seconds, about 180 seconds, about 200 seconds, about 220 seconds, about 240 seconds, about 260 seconds, about 280 seconds, about 300 seconds, about 320 seconds, about 340 seconds, about 360 seconds, about 7 minutes, about 8 minutes, about 9 minutes or about 10 minutes following delivery of nicotine or protonated nicotine in accordance with the teachings of the present disclosure. In embodiments, the effective amount of nicotine or protonated nicotine raises the heart rate of a user by about 10%, or about 15%, or about 20%, or about 25%, or about 30%, or about 35%, or about 40%, or about 45%, or about 50%, or about 55%, or about 60% relative to the heart rate of the user prior to the delivery of nicotine or protonated nicotine in accordance with the teachings of the present disclosure.

The term “respiratory tract” or “respiratory region” as used interchangeably herein and according to the common ordinary meaning, refers to the upper respiratory tract and the lower respiratory tract of a user. The upper respiratory tract includes the nose, nasal cavities, sinuses, pharynx, and the upper portions of the larynx above the vocal folds (e.g., also known as vocal cords or voice reeds.) The lower respiratory tract includes the lower portion of the larynx below the vocal folds, trachea, bronchi, bronchioles and alveoli.

The terms “harsh” and “harshness”, as used interchangeably herein and according to the common ordinary meaning, refer to unpleasant sensory reactions in the respiratory tract, including but not limited to burning or itching sensations and/or other unpleasant or painful sensory reactions in the respiratory tract. Harshness may be quantified, for example, with a visual analogue scale (“VAS”), as well as with other quantification methodologies known in the art such as, for example, where a study participant is asked to rate the level of physical and/or emotional satisfaction he or she felt on a scale of 0-10, with 0 being no physical or emotional satisfaction.

The term “control liquid nicotine formulation” as used herein, refers to an equivalent amount of an acid other than oxalic acid. In embodiments, the control liquid nicotine formulation comprises levulinic acid. In embodiments, the control liquid nicotine formulation comprises benzoic acid. In embodiments, the control liquid nicotine formulation comprises succinic acid. In embodiments, the control liquid nicotine formulation comprises salicylic acid. In embodiments, the control liquid nicotine formulation comprises malic acid. In embodiments, the control liquid nicotine formulation comprises pyruvic acid. In embodiments, the control liquid nicotine formulation comprises citric acid. In embodiments, the control liquid nicotine formulation comprises lauric acid. In embodiments, the control liquid nicotine formulation comprises sorbic acid.

The terms “visual analogue scale” and “VAS”, as used interchangeably herein and according to common and ordinary meaning, refer to a measurement technique that measures a characteristic or attitude that is believed to range across a continuum of values and cannot easily be directly measured. It is often used in epidemiologic and clinical research to measure the intensity or frequency of various symptoms. For example, the amount of pain that a patient feels ranges across a continuum from none to an extreme amount of pain. For example, the pain VAS can be a unidimensional measure of pain intensity, which has been widely used in diverse adult populations, including, for example, those with rheumatic diseases. VAS can be presented in a number of ways, including, but not limited to, scales with a middle point, graduations or numbers (numerical rating scales), meter-shaped scales (curvilinear analogue scales), “box-scales” consisting of circles equidistant from each other (one of which the subject has to mark), and scales with descriptive terms at intervals along a line (graphic rating scales or Likert scales). The VAS can be, for example, a straight horizontal line of fixed length, where the ends are defined as the extreme limits of the parameter to be measured (symptom, pain, health) orientated from one end (worst) to the other end (best). Similarly, horizontal scales can be orientated from right to left and/or can be used vertically. VAS are generally completed by patients themselves but are sometimes used to elicit opinions from health professionals. The patient marks on the line the point that they feel represents their perception of their current state. The VAS score is determined by measuring in some dimension, such as millimeters, from a left hand end of the line to the point that the patient marks. Most commonly, respondents are asked to report “current” pain intensity or pain intensity “in the last 24 hours.” In one example, using a ruler, the score can be determined by measuring the distance (mm) on the 10-cm line between the “no pain” anchor and the patient's mark, providing a range of scores from 0-100. A higher score indicates greater pain intensity. (see for example, http://www.amda.com/tools/library/whitepapers/hospiceinltc/appendix-a.pdf.). A similar VAS scale can be used to measure the potential harshness of the effect of vaporized nicotine substances, including those described herein.

As used in this specification and the claims, unless otherwise stated, the term “about” refers to variations of 1%, 2%, 3%, 4%, 5%, 10%, 15%, or 25%, depending on the embodiment. In embodiments, the term “about” refers to a variation of 1%. In embodiments, the term “about” refers to a variation of 2%. In embodiments, the term “about” refers to a variation of 1%. In embodiments, the term “about” refers to a variation of 3%. In embodiments, the term “about” refers to a variation of 4%. In embodiments, the term “about” refers to a variation of 5%. In embodiments, the term “about” refers to a variation of 6%. In embodiments, the term “about” refers to a variation of 7%. In embodiments, the term “about” refers to a variation of 8%. In embodiments, the term “about” refers to a variation of 9%. In embodiments, the term “about” refers to a variation of 10%.

The terms “suitable biologically compatible carrier” and “biologically acceptable liquid carrier” as used herein include a liquid solvent or medium in which a nicotine salt is soluble (e.g. at ambient conditions, such as 25 degrees Celsius) such that the nicotine salt does not form a solid precipitate. Examples include, but are not limited to, glycerol, propylene glycol, trimethylene glycol, water, ethanol and the like, as well as combinations thereof. For example, the biologically acceptable liquid carrier includes a ratio of propylene glycol and vegetable glycerin. In embodiments, the biologically acceptable liquid carrier comprises 10% to 70% of propylene glycol and 90% to 30% of vegetable glycerin. In some embodiments, the biologically acceptable liquid carrier comprises 20% to 50% of propylene glycol and 80% to 50% of vegetable glycerin. In some embodiments, the biologically acceptable liquid carrier comprises 30% propylene glycol and 70% vegetable glycerin. In other embodiments, the liquid carrier is completely propylene glycol or vegetable glycerin. In other embodiments, the liquid carrier can be other similar aerosol forming agents similar to propylene glycol, glycerin, or other glycols or the like, in various combinations.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the aspects and embodiments described herein. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed as well as intermediate decimal values.

Nicotine is a chemical stimulant and increases, for example, heart rate and blood pressure when provided to an individual or animal. Nicotine transfer to an individual is associated with a feeling of physical and/or emotional satisfaction. Conflicting reports have been published regarding the transfer efficiency of free base nicotine in comparison to mono-or di-protonated nicotine salts. Studies on the transfer efficiency of free base nicotine and nicotine salts are complex and have yielded unpredictable results. Further, such transfer efficiency studies have been performed under extremely high temperature conditions, comparable to smoking; therefore, they may offer little guidance on the transfer efficiency of free base nicotine and nicotine salts under low-temperature vaporization conditions. Some reports have posited that nicotine free base should give rise to a greater satisfaction in a user than any corresponding nicotine salt.

### Methods and Compositions

In an aspect, a method of delivering nicotine to a user of an electronic cigarette is provided. The method comprises (the user) operating an electronic cigarette comprising a liquid nicotine formulation, where the liquid nicotine comprises nicotine, oxalic acid and a biologically acceptable liquid carrier. In accordance with the teachings of the present disclosure, the liquid nicotine formulation is heated to an operating temperature with the electronic cigarette such that the heating delivers an effective amount of nicotine or protonated nicotine

In an aspect, a method of delivering (or administrating) protonated nicotine to a user of an electronic cigarette is provided (also referred to herein as a subject). The method includes (a) (the user) operating an electronic cigarette including a liquid nicotine formulation, the liquid nicotine formulation including nicotine, oxalic acid and a biologically acceptable liquid carrier; (b) heating the liquid nicotine formulation to an operating temperature, such that the heating provides an inhalable aerosol including an effective amount of protonated nicotine; and (c) (the user) inhaling the inhalable aerosol. Operating the electronic cigarette includes activating the essential electronic components of the electronic cigarette to allow for the heating and inhalation.

In an aspect, a method of delivering (or administrating) protonated nicotine to a user of an electronic cigarette is provided (also referred to herein as a subject). The method includes (a) heating the liquid nicotine formulation to an operating temperature using an electronic cigarette including a liquid nicotine formulation, the liquid nicotine formulation including nicotine, oxalic acid and a biologically acceptable liquid carrier, such that the heating provides an inhalable aerosol including an effective amount of protonated nicotine; and (b) (the user) inhaling the inhalable aerosol.

In another aspect, a liquid nicotine formulation is provided including nicotine, oxalic acid, and a biologically acceptable liquid carrier. In embodiments, when heating the liquid nicotine formulation, an inhalable aerosol is formed comprising an effective amount of nicotine or protonated nicotine. In embodiments, when heating the liquid nicotine formulation, an inhalable aerosol is formed comprising an effective amount of protonated nicotine. In embodiments, the liquid nicotine formulation is in a cartridge. In embodiments, the cartridge is in an electronic cigarette.

In embodiments, the liquid nicotine formulation does not include any organic acid other than oxalic acid. In embodiments, the liquid nicotine formulation does not include any acid other than oxalic acid.

In embodiments, the delivery of an effective amount of nicotine or protonated nicotine from the liquid nicotine formulation results in the user experiencing less respiratory tract harshness relative to a control liquid nicotine formulation comprising an acid other than oxalic acid. In embodiments, the delivery of an effective amount of protonated nicotine from the liquid nicotine formulation results in the user experiencing less respiratory tract harshness relative to a control liquid nicotine formulation comprising an acid other than oxalic acid. In embodiments, the control liquid nicotine formulation includes a molar amount of levulinic acid and nicotine corresponding to the molar amount of oxalic acid and nicotine in the liquid nicotine formulation. In embodiments, the control liquid nicotine formulation includes a molar amount of benzoic acid and nicotine corresponding to the molar amount of oxalic acid and nicotine in the liquid nicotine formulation. In embodiments, the control liquid nicotine formulation includes a molar amount of succinic acid and nicotine corresponding to the molar amount of oxalic acid and nicotine in the liquid nicotine formulation. In embodiments, the control liquid nicotine formulation includes a molar amount of salicylic acid and nicotine corresponding to the molar amount of oxalic acid and nicotine in the liquid nicotine formulation. In embodiments, the control liquid nicotine formulation includes a molar amount of malic acid and nicotine corresponding to the molar amount of oxalic acid and nicotine in the liquid nicotine formulation. In embodiments, the control liquid nicotine formulation includes a molar amount of pyruvic acid and nicotine corresponding to the molar amount of oxalic acid and nicotine in the liquid nicotine formulation. In embodiments, the control liquid nicotine formulation includes a molar amount of citric acid and nicotine corresponding to the molar amount of oxalic acid and nicotine in the liquid nicotine formulation. In embodiments, the control liquid nicotine formulation includes a molar amount of lauric acid and nicotine corresponding to the molar amount of oxalic acid and nicotine in the liquid nicotine formulation. In embodiments, the control liquid nicotine formulation includes a molar amount of sorbic acid and nicotine corresponding to the molar amount of oxalic acid and nicotine in the liquid nicotine formulation.

In embodiments, the user experiences less respiratory harshness in the upper respiratory tract as compared to a control liquid nicotine formulation. In embodiments, the user experiences less harshness in the nose as compared to a control liquid nicotine formulation. In embodiments, the user experiences less harshness in the nasal cavities as compared to a control liquid nicotine formulation. In embodiments, the user experiences less harshness in the sinuses as compared to a control liquid nicotine formulation. In embodiments, the user experiences less harshness in the pharynx as compared to a control liquid nicotine formulation. In embodiments, the user experiences less harshness in the upper portions of the larynx as compared to a control liquid nicotine formulation. In embodiments, the user experiences less harshness in the lower portions of the larynx as compared to a control liquid nicotine formulation. In embodiments, the user experiences less harshness in the trachea as compared to a control liquid nicotine formulation. In embodiments, the user experiences less harshness in the bronchi as compared to a control liquid nicotine formulation. In embodiments, the user experiences less harshness in the bronchioles as compared to a control liquid nicotine formulation. In embodiments, the user experiences less harshness in the alveoli as compared to a control liquid nicotine formulation.

In embodiments, the delivery of an effective amount of protonated nicotine from the liquid nicotine formulation results in the user experiencing more than about 5% less harshness compared to a control liquid nicotine formulation. In embodiments, the delivery of an effective amount of protonated nicotine from the liquid nicotine formulation results in the user experiencing more than about 10% less harshness compared to a control liquid nicotine formulation. In embodiments, the delivery of an effective amount of protonated nicotine from the liquid nicotine formulation results in the user experiencing more than about 15% less harshness compared to a control liquid nicotine formulation. In embodiments, the delivery of an effective amount of protonated nicotine from the liquid nicotine formulation results in the user experiencing more than about 20% less harshness compared to a control liquid nicotine formulation. In embodiments, the delivery of an effective amount of protonated nicotine from the liquid nicotine formulation results in the user experiencing more than about 25% less harshness compared to a control liquid nicotine formulation. In embodiments, the delivery of an effective amount of protonated nicotine from the liquid nicotine formulation results in the user experiencing more than about 30% less harshness compared to a control liquid nicotine formulation. In embodiments, the delivery of an effective amount of protonated nicotine from the liquid nicotine formulation results in the user experiencing more than about 35% less harshness compared to a control liquid nicotine formulation. In embodiments, the delivery of an effective amount of protonated nicotine from the liquid nicotine formulation results in the user experiencing more than about 40% less harshness compared to a control liquid nicotine formulation. In embodiments, the delivery of an effective amount of protonated nicotine from the liquid nicotine formulation results in the user experiencing more than about 45% less harshness compared to a control liquid nicotine formulation. In embodiments, the delivery of an effective amount of protonated nicotine from the liquid nicotine formulation results in the user experiencing more than about 50% less harshness compared to a control liquid nicotine formulation. In embodiments, the delivery of an effective amount of protonated nicotine from the liquid nicotine formulation results in the user experiencing more than about 55% less harshness compared to a control liquid nicotine formulation. In embodiments, the delivery of an effective amount of protonated nicotine from the liquid nicotine formulation results in the user experiencing more than about 60% less harshness compared to a control liquid nicotine formulation. In embodiments, the delivery of an effective amount of protonated nicotine from the liquid nicotine formulation results in the user experiencing more than about 65% less harshness compared to a control liquid nicotine formulation. In embodiments, the delivery of an effective amount of protonated nicotine from the liquid nicotine formulation results in the user experiencing more than about 70% less harshness compared to a control liquid nicotine formulation. In embodiments, the delivery of an effective amount of protonated nicotine from the liquid nicotine formulation results in the user experiencing more than about 75% less harshness compared to a control liquid nicotine formulation. In embodiments, the delivery of an effective amount of protonated nicotine from the liquid nicotine formulation results in the user experiencing more than about 80% less harshness compared to a control liquid nicotine formulation. In embodiments, the delivery of an effective amount of protonated nicotine from the liquid nicotine formulation results in the user experiencing more than about 85% less harshness compared to a control liquid nicotine formulation. In embodiments, the delivery of an effective amount of protonated nicotine from the liquid nicotine formulation results in the user experiencing more than about 90% less harshness compared to a control liquid nicotine formulation. In embodiments, the delivery of an effective amount of protonated nicotine from the liquid nicotine formulation results in the user experiencing more than about 95% less harshness compared to a control liquid nicotine formulation. In embodiments, the delivery of an effective amount of protonated nicotine from the liquid nicotine formulation results in the user experiencing about 100% less harshness compared to a control liquid nicotine formulation.

In embodiments, the delivery of an effective amount of protonated nicotine from the liquid nicotine formulation results in the user experiencing about 5% less harshness compared to a control liquid nicotine formulation. In embodiments, the delivery of an effective amount of protonated nicotine from the liquid nicotine formulation results in the user experiencing about 10% less harshness compared to a control liquid nicotine formulation. In embodiments, the delivery of an effective amount of protonated nicotine from the liquid nicotine formulation results in the user experiencing about 15% less harshness compared to a control liquid nicotine formulation. In embodiments, the delivery of an effective amount of protonated nicotine from the liquid nicotine formulation results in the user experiencing about 20% less harshness compared to a control liquid nicotine formulation. In embodiments, the delivery of an effective amount of protonated nicotine from the liquid nicotine formulation results in the user experiencing about 25% less harshness compared to a control liquid nicotine formulation. In embodiments, the delivery of an effective amount of protonated nicotine from the liquid nicotine formulation results in the user experiencing about 30% less harshness compared to a control liquid nicotine formulation. In embodiments, the delivery of an effective amount of protonated nicotine from the liquid nicotine formulation results in the user experiencing about 35% less harshness compared to a control liquid nicotine formulation. In embodiments, the delivery of an effective amount of protonated nicotine from the liquid nicotine formulation results in the user experiencing about 40% less harshness compared to a control liquid nicotine formulation. In embodiments, the delivery of an effective amount of protonated nicotine from the liquid nicotine formulation results in the user experiencing about 45% less harshness compared to a control liquid nicotine formulation. In embodiments, the delivery of an effective amount of protonated nicotine from the liquid nicotine formulation results in the user experiencing about 50% less harshness compared to a control liquid nicotine formulation. In embodiments, the delivery of an effective amount of protonated nicotine from the liquid nicotine formulation results in the user experiencing about 55% less harshness compared to a control liquid nicotine formulation. In embodiments, the delivery of an effective amount of protonated nicotine from the liquid nicotine formulation results in the user experiencing about 60% less harshness compared to a control liquid nicotine formulation. In embodiments, the delivery of an effective amount of protonated nicotine from the liquid nicotine formulation results in the user experiencing about 65% less harshness compared to a control liquid nicotine formulation. In embodiments, the delivery of an effective amount of protonated nicotine from the liquid nicotine formulation results in the user experiencing about 70% less harshness compared to a control liquid nicotine formulation. In embodiments, the delivery of an effective amount of protonated nicotine from the liquid nicotine formulation results in the user experiencing about 75% less harshness compared to a control liquid nicotine formulation. In embodiments, the delivery of an effective amount of protonated nicotine from the liquid nicotine formulation results in the user experiencing about 80% less harshness compared to a control liquid nicotine formulation. In embodiments, the delivery of an effective amount of protonated nicotine from the liquid nicotine formulation results in the user experiencing about 85% less harshness compared to a control liquid nicotine formulation. In embodiments, the delivery of an effective amount of protonated nicotine from the liquid nicotine formulation results in the user experiencing about 90% less harshness compared to a control liquid nicotine formulation. In embodiments, the delivery of an effective amount of protonated nicotine from the liquid nicotine formulation results in the user experiencing about 95% less harshness compared to a control liquid nicotine formulation. In embodiments, the delivery of an effective amount of protonated nicotine from the liquid nicotine formulation results in the user experiencing about 100% less harshness compared to a control liquid nicotine formulation.

In embodiments, the delivery of an effective amount of protonated nicotine from the liquid nicotine formulation results in the user experiencing about 5% to about 50% less harshness compared to a control liquid nicotine formulation. In embodiments, the delivery of an effective amount of protonated nicotine from the liquid nicotine formulation results in the user experiencing about 10% to about 45% less harshness as compared to a control liquid nicotine formulation. In embodiments, the delivery of an effective amount of protonated nicotine from the liquid nicotine formulation results in the user experiencing about 15% to about 40% less harshness as compared to a control liquid nicotine formulation. In embodiments, the delivery of an effective amount of protonated nicotine from the liquid nicotine formulation results in the user experiencing about 20% to about 35% less harshness as compared to a control liquid nicotine formulation. In embodiments, the delivery of an effective amount of protonated nicotine from the liquid nicotine formulation results in the user experiencing about 5% to about 100% less harshness compared to a control liquid nicotine formulation. In embodiments, the delivery of an effective amount of protonated nicotine from the liquid nicotine formulation results in the user experiencing about 5% to about 95% less harshness compared to a control liquid nicotine formulation. In embodiments, the delivery of an effective amount of protonated nicotine from the liquid nicotine formulation results in the user experiencing about 5% to about 90% less harshness compared to a control liquid nicotine formulation. In embodiments, the delivery of an effective amount of protonated nicotine from the liquid nicotine formulation results in the user experiencing about 5% to about 80% less harshness compared to a control liquid nicotine formulation. In embodiments, the delivery of an effective amount of protonated nicotine from the liquid nicotine formulation results in the user experiencing about 5% to about 70% less harshness compared to a control liquid nicotine formulation. In embodiments, the delivery of an effective amount of protonated nicotine from the liquid nicotine formulation results in the user experiencing about 5% to about 60% less harshness compared to a control liquid nicotine formulation.

In embodiments, the nicotine is protonated. In embodiments, the number or moles of oxalic acid functional groups is equal to or greater than the molar amount of nicotine. In embodiments, the number or moles of oxalic acid functional groups is equal to the molar amount of nicotine.

In embodiments, the number or moles of oxalic acid functional groups is greater than the molar amount of nicotine.

In embodiments, the number or moles of oxalic acid functional groups is from about 1.1 times greater to about 3.0 times greater than the molar amount of nicotine. In embodiments, the number of oxalic acid functional groups is from about 1.5 times greater to about 2.2 times greater than the molar amount of nicotine.

In embodiments, the amount of or moles of excess oxalic acid functional groups is about 1.1 times greater, or about 1.2 times greater, or about 1.3 times greater, or about 1.4 times greater, or about 1.5 times greater, or about 1.6 times greater, or about 1.7 times greater, or about 1.8 times greater, or about 2 times greater, or about 2.1 times greater, or about 2.2 times greater, or about 2.3 times greater, or about 2.4 times greater, or about 2.5 times greater, or about 2.6 times greater, or about 2.7 times greater, or about 2.8 times greater, or about 2.9 times greater, or about 3.0 times greater, etc., than the molar amount of nicotine present in the liquid nicotine formulation. In embodiments, the excess amount or moles of oxalic acid functional groups, provide less harshness upon inhalation to a user relative to a control liquid nicotine formulation.

In embodiments, the molar ratio of oxalic acid to nicotine is about 0.5:1. In embodiments, the molar ratio of oxalic acid to nicotine is about 0.6:1. In embodiments, the molar ratio of oxalic acid to nicotine is about 0.7:1. In embodiments, the molar ratio of oxalic acid to nicotine is about 0.8:1. In embodiments, the molar ratio of oxalic acid to nicotine is about 0.9:1. In embodiments, the molar ratio of oxalic acid to nicotine is about 1.0:1. In embodiments, the molar ratio of oxalic acid to nicotine is about 1.1:1. In embodiments, the molar ratio of oxalic acid to nicotine is about 1.2:1. In embodiments, the molar ratio of oxalic acid to nicotine is about 1.3:1. In embodiments, the molar ratio of oxalic acid to nicotine is about 1.4:1. In embodiments, the molar ratio of oxalic acid to nicotine is about 1.5:1. In embodiments, the molar ratio of oxalic acid to nicotine is about 1.6:1. In embodiments, the molar ratio of oxalic acid to nicotine is about 1.7:1. In embodiments, the molar ratio of oxalic acid to nicotine is about 1.8:1. In embodiments, the molar ratio of oxalic acid to nicotine is about 1.9:1. In embodiments, the molar ratio of oxalic acid to nicotine is about 2.0:1. In embodiments, the molar ratio of oxalic acid to nicotine is about 3:1. In embodiments, the molar ratio of oxalic acid to nicotine is about 4:1. In embodiments, the molar ratio of oxalic acid to nicotine is about 5:1. In embodiments, the molar ratio of oxalic acid to nicotine is about 6:1. In embodiments, the molar ratio of oxalic acid to nicotine is about 7:1. In embodiments, the molar ratio of oxalic acid to nicotine is about 8:1. In embodiments, the molar ratio of oxalic acid to nicotine is about 9:1. In embodiments, the molar ratio of oxalic acid to nicotine is about 10:1. In embodiments, the molar ratio of oxalic acid to nicotine is about 11:1. In embodiments, the molar ratio of oxalic acid to nicotine is about 12:1. In embodiments, the molar ratio of oxalic acid to nicotine is about 13:1. In embodiments, the molar ratio of oxalic acid to nicotine is about 14:1. In embodiments, the molar ratio of oxalic acid to nicotine is about 15:1. In embodiments, the molar ratio of oxalic acid to nicotine is about 16:1. In embodiments, the molar ratio of oxalic acid to nicotine is about 17:1. In embodiments, the molar ratio of oxalic acid to nicotine is about 18:1. In embodiments, the molar ratio of oxalic acid to nicotine is about 19:1. In embodiments, the molar ratio of oxalic acid to nicotine is about 20:1.

In embodiments, the molar ratio of oxalic acid to nicotine is at least 0.5:1. In embodiments, the molar ratio of oxalic acid to nicotine is at least 0.6:1. In embodiments, the molar ratio of oxalic acid to nicotine is at least 0.7:1. In embodiments, the molar ratio of oxalic acid to nicotine is at least 0.8:1. In embodiments, the molar ratio of oxalic acid to nicotine is at least 0.9:1. In embodiments, the molar ratio of oxalic acid to nicotine is at least 1.0:1. In embodiments, the molar ratio of oxalic acid to nicotine is at least 1.1:1. In embodiments, the molar ratio of oxalic acid to nicotine is at least 1.2:1. In embodiments, the molar ratio of oxalic acid to nicotine is at least 1.3:1. In embodiments, the molar ratio of oxalic acid to nicotine is at least 1.4:1. In embodiments, the molar ratio of oxalic acid to nicotine is at least 1.5:1. In embodiments, the molar ratio of oxalic acid to nicotine is at least 1.6:1. In embodiments, the molar ratio of oxalic acid to nicotine is at least 1.7:1. In embodiments, the molar ratio of oxalic acid to nicotine is at least 1.8:1. In embodiments, the molar ratio of oxalic acid to nicotine is at least 1.9:1. In embodiments, the molar ratio of oxalic acid to nicotine is at least 2.0:1. In embodiments, the molar ratio of oxalic acid to nicotine is at least 3:1. In embodiments, the molar ratio of oxalic acid to nicotine is at least 4:1. In embodiments, the molar ratio of oxalic acid to nicotine is at least 5:1. In embodiments, the molar ratio of oxalic acid to nicotine is at least 6:1. In embodiments, the molar ratio of oxalic acid to nicotine is at least 7:1. In embodiments, the molar ratio of oxalic acid to nicotine is at least 8:1. In embodiments, the molar ratio of oxalic acid to nicotine is at least 9:1. In embodiments, the molar ratio of oxalic acid to nicotine is at least 10:1. In embodiments, the molar ratio of oxalic acid to nicotine is at least 11:1. In embodiments, the molar ratio of oxalic acid to nicotine is at least 12:1. In embodiments, the molar ratio of oxalic acid to nicotine is at least 13:1. In embodiments, the molar ratio of oxalic acid to nicotine is at least 14:1. In embodiments, the molar ratio of oxalic acid to nicotine is at least 15:1. In embodiments, the molar ratio of oxalic acid to nicotine is at least 16:1. In embodiments, the molar ratio of oxalic acid to nicotine is at least 17:1. In embodiments, the molar ratio of oxalic acid to nicotine is at least 18:1. In embodiments, the molar ratio of oxalic acid to nicotine is at least 19:1. In embodiments, the molar ratio of oxalic acid to nicotine is at least 20:1.

In embodiments, the nicotine concentration in the liquid nicotine formulation is from about 0.5% (w/w) to about 20% (w/w). In embodiments, the nicotine concentration in the liquid nicotine formulation is from about 1.0% (w/w) to about 15% (w/w). In embodiments, the nicotine concentration in the liquid nicotine formulation is from about 2.0% (w/w) to about 10% (w/w). In embodiments, the nicotine concentration in the liquid nicotine formulation is from about 3.0% (w/w) to about 8% (w/w). In embodiments, the nicotine concentration in the liquid nicotine formulation is from about 3.5% (w/w) to about 7% (w/w). In embodiments, the nicotine concentration in the liquid nicotine formulation is from about 4% (w/w) to about 6% (w/w). In embodiments, the nicotine concentration in the liquid nicotine formulation is about 0.5% (w/w). In embodiments, the nicotine concentration in the liquid nicotine formulation is about 1.0% (w/w). In embodiments, the nicotine concentration in the liquid nicotine formulation is about 1.5% (w/w). In embodiments, the nicotine concentration in the liquid nicotine formulation is about 2.0% (w/w). In embodiments, the nicotine concentration in the liquid nicotine formulation is about 2.5% (w/w). In embodiments, the nicotine concentration in the liquid nicotine formulation is about 3.0% (w/w). In embodiments, the nicotine concentration in the liquid nicotine formulation is about 3.5% (w/w). In embodiments, the nicotine concentration in the liquid nicotine formulation is about 4.0% (w/w). In embodiments, the nicotine concentration in the liquid nicotine formulation is about 4.5% (w/w). In embodiments, the nicotine concentration in the liquid nicotine formulation is about 5.0% (w/w). In embodiments, the nicotine concentration in the liquid nicotine formulation is about 5.5% (w/w). In embodiments, the nicotine concentration in the liquid nicotine formulation is about 6.0% (w/w). In embodiments, the nicotine concentration in the liquid nicotine formulation is about 6.5% (w/w). In embodiments, the nicotine concentration in the liquid nicotine formulation is about 7.0% (w/w). Certain embodiments provide a liquid nicotine formulation having a nicotine concentration of about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15%, 16%, 17%, 18%, 19%, or 20% (w/w),), or more, including any increments therein.

In embodiments, the oxalic acid concentration is from about 2.5% to about 3.5% oxalic acid. In embodiments, the liquid nicotine formulation includes about 4% to about 5% nicotine (w/w) and about 2.5% to about 3.5% oxalic acid (w/w). In embodiments, the liquid nicotine formulation includes about 5% nicotine (w/w) and about 2.8% (w/w) oxalic acid.

In embodiments, the liquid nicotine formulation includes about 0.5% (w/w) oxalic acid. In embodiments, the liquid nicotine formulation includes about 1.0% (w/w) oxalic acid. In embodiments, the liquid nicotine formulation includes about 1.5% (w/w) oxalic acid. In embodiments, the liquid nicotine formulation includes about 2.0% (w/w) oxalic acid. In embodiments, the liquid nicotine formulation includes about 2.5% (w/w) oxalic acid. In embodiments, the liquid nicotine formulation includes about 3.0% (w/w) oxalic acid. In embodiments, the liquid nicotine formulation includes about 3.5% (w/w) oxalic acid. In embodiments, the liquid nicotine formulation includes about 4.0% (w/w) oxalic acid. In embodiments, the liquid nicotine formulation includes about 4.5% (w/w) oxalic acid. In embodiments, the liquid nicotine formulation includes about 5.0% (w/w) oxalic acid. In embodiments, the liquid nicotine formulation includes from about 2.5% to about 3.5% oxalic acid. In embodiments, the liquid nicotine formulation includes from about 2.0% to about 4.0% oxalic acid. In embodiments, the liquid nicotine formulation includes from about 1.5% to about 4.5% oxalic acid. In embodiments, the liquid nicotine formulation includes from about 1.0% to about 4.5% oxalic acid. In embodiments, the liquid nicotine formulation includes about 2.6% oxalic acid. In embodiments, the liquid nicotine formulation includes about 2.7% oxalic acid. In embodiments, the liquid nicotine formulation includes about 2.8% oxalic acid. In embodiments, the liquid nicotine formulation includes about 2.9% oxalic acid. In embodiments, the liquid nicotine formulation includes about 3.1% oxalic acid. In embodiments, the liquid nicotine formulation includes about 3.2% oxalic acid. In embodiments, the liquid nicotine formulation includes about 3.3% oxalic acid. In embodiments, the liquid nicotine formulation includes about 3.4% oxalic acid.

In embodiments, the liquid nicotine formulation includes a molar excess of oxalic acid relative to the amount of nicotine.

In embodiments, the excess of oxalic acid reduces the harshness of the inhalable aerosol produced from the liquid nicotine formulation in the upper respiratory tract and/or lower respiratory tract of the user. Thus, in embodiments, the excess oxalic acid may prevent irritation and/or enhance smoothness. In embodiments, the excess oxalic acid sustains protonation of the nicotine after it has been delivered (e.g. into the upper respiratory region. In embodiments, where the nicotine causes irritation, the excess oxalic acid sustains protonation of nicotine thereby reducing harshness.

Oxalic acid has 2 acid functional groups (—COOH groups), each or which is also referred to herein as an oxalic acid functional group. Thus, oxalic acid provides twice the protonation capability per mole relative to an organic acid with one acid functional group (e.g. one —COOH). Thus, in embodiments, an enhanced smoothness and/or reduced harshness with deep lung delivery may be achieved with a liquid nicotine formulation of about 5% nicotine and about 2.8% oxalic acid by weight (+/0.2% oxalic acid).

Described herein are nicotine formulations and electronic cigarettes (vaporizers) including the liquid nicotine these formulations of the present disclosure. In particular, described herein are liquid solutions for use in an electronic vaporizer, and particularly vaporizers having a resistive heating element (e.g., coil) and a wick. These formulations typically include a liquid solution of a nicotine salt formulated with oxalic acid to protonate the nicotine. In embodiments, the use of excess of oxalic acid results in a user experience that exceeds what would be expected using other organic acids having carboxylic acids, and further may also be more readily compatible for use with a wicked liquid nicotine solution.

In embodiment, the nicotine and oxalic acid formulations described herein provide satisfaction in an individual superior to that of free base nicotine, and more comparable to the satisfaction in an individual smoking a traditional cigarette. In embodiments, the satisfaction effect is consistent with an efficient transfer of nicotine to the lungs of an individual and a rapid rise of nicotine absorption in the plasma similar to that shown, for example, in Example 8. Therefore, described herein are formulations containing nicotine and oxalic acid for use in an electronic cigarette, or the like, that provide a general satisfaction effect consistent with an efficient transfer of nicotine to the lungs of an individual and a rapid rise of nicotine absorption in the plasma. Provided herein, therefore, are devices, formulation of nicotine salts, systems, cartomizers, kits and methods that are used to inhale an aerosol generated from a liquid nicotine formulation through the mouth or nose as described herein or as would be apparent to one of skill in the art upon reading the disclosure herein.

In embodiments, there is a difference between the Cₘₐₓ (maximum concentration) and Tₘₐₓ (time at which the maximum concentration is measured) when measuring blood plasma nicotine levels of freebase nicotine formulations inhaled using a low temperature vaporization device, i.e. electronic cigarette, as compared to the Cₘₐₓ and Tₘₐₓ (similarly measuring blood plasma nicotine levels) of a traditional cigarette. In embodiments, it has unexpectedly been found herein that there is a difference between the Cₘₐₓ (maximum concentration) and Tₘₐₓ (time at which the maximum concentration is measured) when measuring blood plasma nicotine levels of freebase nicotine formulations inhaled using a low temperature vaporization device, i.e. electronic cigarette, as compared to the Cₘₐₓ and Tₘₐₓ (similarly measuring blood plasma nicotine levels) of nicotine salt formulations inhaled using a low temperature vaporization device, i.e. electronic cigarette. In embodiments, there is a difference between the rate of nicotine uptake in the plasma of users inhaling freebase nicotine formulations using a low temperature vaporization device, i.e. electronic cigarette, as compared to the rate of nicotine uptake in the plasma of users inhaling smoke of a traditional cigarette. In embodiments, there is a difference between the rate of nicotine uptake in the plasma of users inhaling freebase nicotine formulations using a low temperature vaporization device, i.e. electronic cigarette, as compared to the rate of nicotine uptake in the plasma of users inhaling nicotine salt formulations using a low temperature vaporization device, i.e. electronic cigarette.

Thus, looking at freebase nicotine as a source of nicotine in compositions used in e-cigarettes, freebase nicotine compositions' delivery of nicotine to blood when inhaled using is not necessarily comparable in blood plasma levels (Cₘₐₓ and Tₘₐₓ) to a traditional cigarette's nicotine delivery to blood when inhaled. Freebase nicotine compositions' delivery of nicotine to blood when inhaled using is not necessarily comparable in blood plasma levels (Cₘₐₓ and Tₘₐₓ) to nicotine salt formulations' nicotine delivery to blood when inhaled. Freebase nicotine compositions' delivery of nicotine to blood when inhaled using is not necessarily comparable in blood plasma levels when measuring the rate of nicotine uptake in the blood within the first 0-5 minutes to a traditional cigarette's nicotine delivery to blood when inhaled. Freebase nicotine compositions' delivery of nicotine to blood when inhaled using necessarily is not comparable in blood plasma levels when measuring the rate of nicotine uptake in the blood within the first 0-5 minutes to nicotine salt formulations' nicotine delivery to blood when inhaled.

In embodiments, there appears to be comparable Cₘₐₓ and Tₘₐₓ values (measuring blood plasma nicotine levels) of nicotine-oxalic acid formulations inhaled using a low temperature vaporization device, i.e. electronic cigarette, as compared to the Cₘₐₓ and Tₘₐₓ (similarly measuring blood plasma nicotine levels) of a traditional cigarette, there is a demonstrable difference between the rate of nicotine uptake in the plasma of users inhaling certain nicotine salt formulations using a low temperature vaporization device, i.e. electronic cigarette, as compared to the rate of nicotine uptake in the plasma of users inhaling other nicotine salt formulations using a low temperature vaporization device, i.e. electronic cigarette. In embodiments, the Cₘₐₓ and Tₘₐₓ values are comparable to those of a traditional cigarette, (or are approaching that of a traditional cigarette), the rate of nicotine uptake in the plasma of blood of users is higher in certain nicotine salt formulations than that of the traditional cigarette. The nicotine-oxalic acid formulations which demonstrate the quickest rate of nicotine uptake in the plasma are more preferred in satisfaction evaluations, and are rated more equivalent to cigarette satisfaction than the nicotine salt formulations showing the slowest rates of rise of nicotine in the subjects' blood plasma. In embodiments, doubling the concentration of the nicotine in the formulation may not necessarily impact the rate of absorption of nicotine in the blood (see, for an example, Example 8, nicotine benzoate tested in 4% and 2% concentrations).

In embodiments, nicotine-oxalic acid formulations delivered using an e-cigarette are comparable in Cₘₐₓ and Tₘₐₓ values (measuring blood plasma nicotine levels). In embodiments, nicotine-oxalic acid formulations include one or more additional organic acids having a Vapor Pressure between 20-300 mmHg @ 200° C., or Vapor Pressure>20 mmHg @ 200° C., or a Vapor Pressure from 20 to 300 mmHg @ 200° C., or a Vapor Pressure from 20 to 200 mmHg @ 200° C. Organic acids with a Vapor Pressure between 20 and 300 mmHg @ 200° C. appear to have a higher rate of nicotine uptake in the blood at early time periods (0-1.5 minutes, 0-3 minutes, 0-2 minutes, 0-4 minutes for non-limiting example) compared to other nicotine acid formulations, however, they also provide satisfaction comparable to a traditional cigarette or closer to a traditional cigarette (as compared to other nicotine salt formulations or as compared to nicotine freebase formulations). For non-limiting example, additional organic acids that meet one or more criteria of the prior sentence include salicylic acid, sorbic acid, benzoic acid, lauric acid, and levulinic acid. In embodiments, nicotine-oxalic acid salt formulations made using additional organic acids that have a difference between boiling point and melting point of at least 50° C., and a boiling point greater than 160° C., and a melting point less than 160° C. have a higher rate of nicotine uptake in the blood at early time periods (0-1.5 minutes, 0-3 minutes, 0-2 minutes, 0-4 minutes for non-limiting example) than formulations using other types of additional organic acids and/or may provide satisfaction comparable to a traditional cigarette or closer to a traditional cigarette (as compared to other nicotine salt formulations or as compared to nicotine freebase formulations). For non-limiting example, additional acids that meet the criteria of the prior sentence include salicylic acid, sorbic acid, benzoic acid, pyruvic acid, lauric acid, and levulinic acid.

Operating temperature can be 100° C. to 300° C., or about 200° C., about 150° C. to about 250° C., 180C to 220° C., about 180° C. to about 220° C., 185° C. to 215° C., about 185° C. to about 215° C., about 190° C. to about 210° C., 190° C. to 210° C., 195° C. to 205° C., or about 195° C. to about 205° C. In embodiments, additional acids that meet the criteria of the prior sentence include salicylic acid, sorbic acid, benzoic acid, pyruvic acid, lauric acid, and levulinic acid. Combinations of these criteria for preference of certain nicotine-oxalic acid formulations are contemplated herein.

Other reasons for excluding certain additional acids from formulations may be unrelated to the rate of nicotine uptake, however. For example, an additional acid may be inappropriate for use with the device materials (corrosive or otherwise incompatible). Sulfuric acid is an example of this, which may be inappropriate for the e-cigarette device. An acid may be inappropriate for use in inhalation or for toxicity reasons—thus not be compatible for human consumption, ingestion, or inhalation. Sulfuric acid again is an example of this, which may be inappropriate for a user of an e-cigarette device, depending on the embodiment of the composition. An acid that is bitter or otherwise bad-tasting may also provide a reason for exclusion, such as acetic acid in some embodiments. Acids that oxidize at room temperature or at operating temperature may be inappropriate for certain embodiments, for example, sorbic acid, as this indicates a decomposition or reaction or instability that may be undesirable in the formulation. Decomposition of acids at room or operating temperatures may also indicate that the acid is inappropriate for use in the embodiment formulations. For example, citric acid decomposes at 175° C., and malic acid decomposes at 140° C., thus for a device operating at 200° C., these acids may not be appropriate. Acids that have poor solubility in the composition constituents may be inappropriate for use in certain embodiments of the compositions herein. For example, nicotine bitartrate with a composition of nicotine and tartaric acid as 1:2 molar ratio will not produce a solution at a concentration of 0.5% (w/w) nicotine or higher and 0.9% (w/w) tartaric acid or higher in propylene glycol (PG) or vegetable glycerin (VG) or any mixture of PG and VG at ambient conditions. As used herein, weight percentage (w/w) refers to the weight of the individual component over the weight of the total formulation.

Thus, provided herein is a method of delivering nicotine to a user comprising operating an electronic cigarette to a user wherein the electronic cigarette comprises a liquid nicotine formulation comprising nicotine and oxalic acid in a biologically acceptable liquid carrier wherein an additional acid used to form a protonated nicotine is characterized by vapor pressure >20 mmHg at 200° C., and inhaling an aerosol generated from the liquid nicotine formulation heated by the electronic cigarette.

Provided herein is a method of delivering nicotine to a user comprising operating an electronic cigarette to a user wherein the electronic cigarette comprises a liquid nicotine formulation comprising nicotine and oxalic acid in a biologically acceptable liquid carrier. An additional acid may be used that is characterized by vapor pressure of about 20 to 200 mmHg at 200° C., and inhaling an aerosol generated from the liquid nicotine formulation heated by the electronic cigarette.

Provided herein is a method of delivering nicotine to a user comprising operating an electronic cigarette wherein the electronic cigarette comprises a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point, and inhaling an aerosol generated from the liquid nicotine formulation heated by the electronic cigarette.

Provided herein is a method of delivering nicotine to a user comprising providing an electronic cigarette to a user wherein the electronic cigarette comprises a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and inhaling an aerosol generated from the nicotine salt formulation heated by the electronic cigarette.

Provided herein is a method of delivering nicotine to the blood of a user, said method comprising providing an aerosol that is inhaled by the user from an electronic cigarette that comprises a liquid nicotine formulation comprising nicotine and oxalic acid in a biologically acceptable liquid carrier wherein providing the aerosol comprises the electronic cigarette heating the formulation thereby generating the aerosol, wherein the aerosol is effective in delivering a level of nicotine in the blood of the user that is at least 5 ng/mL at about 1.5 minutes after a first puff of ten puffs of the aerosol, each puff taken at 30 second intervals.

Provided herein is a liquid nicotine formulation in an electronic cigarette for generating an inhalable aerosol upon heating in the electronic cigarette, the formulation in the cigarette comprising a liquid nicotine formulation comprising nicotine and oxalic acid in a biologically acceptable liquid carrier. An additional acid may be used that is characterized by vapor pressure >20 mmHg at 200° C.

Provided herein is a liquid nicotine formulation in an electronic cigarette for generating an inhalable aerosol upon heating in the electronic cigarette, the formulation in the cigarette comprising a liquid nicotine formulation comprising nicotine and oxalic acid in a biologically acceptable liquid carrier. An additional acid may be used that is characterized by vapor pressure of about 20 to 200 mmHg at 200° C.

Provided herein is a liquid nicotine formulation in an electronic cigarette for generating an inhalable aerosol upon heating in the electronic cigarette, the formulation in the cigarette comprising a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point.

Provided herein is a liquid nicotine formulation in an electronic cigarette for generating an inhalable aerosol upon heating in the electronic cigarette, the formulation in the cigarette comprising a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a liquid nicotine formulation for generating an inhalable aerosol upon heating in the electronic cigarette, the liquid nicotine formulation comprising a liquid nicotine formulation comprising nicotine and oxalic acid in a biologically acceptable liquid carrier. An additional acid may be used that is characterized by vapor pressure >20 mmHg at 200° C.

Provided herein is a liquid nicotine formulation for generating an inhalable aerosol upon heating in the electronic cigarette, the liquid nicotine formulation comprising a liquid nicotine formulation comprising nicotine and oxalic acid in a biologically acceptable liquid carrier. An additional acid may be used that is characterized by vapor pressure of about 20 to 200 mmHg at 200° C.

Provided herein is a liquid nicotine formulation for generating an inhalable aerosol upon heating in the electronic cigarette, the liquid nicotine formulation comprising a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point.

Provided herein is a liquid nicotine formulation for generating an inhalable aerosol upon heating in the electronic cigarette, the liquid nicotine formulation comprising a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a liquid nicotine formulation for use in an electronic cigarette the liquid nicotine formulation comprising a liquid nicotine formulation comprising nicotine and oxalic acid in a biologically acceptable liquid carrier. An additional acid may be used that is characterized by vapor pressure >20 mmHg at 200° C.

Provided herein is a liquid nicotine formulation for use in an electronic cigarette the liquid nicotine formulation comprising nicotine and oxalic acid in a biologically acceptable liquid carrier. An additional acid may be used that is characterized by vapor pressure of about 20 to 200 mmHg at 200° C.

Provided herein is a liquid nicotine formulation for use in an electronic cigarette the liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point.

Provided herein is a liquid nicotine formulation for use in an electronic cigarette the liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a use of a liquid nicotine formulation for delivery of nicotine to a user from an electronic cigarette wherein the liquid nicotine formulation comprises a liquid nicotine formulation comprising nicotine and oxalic acid in a biologically acceptable liquid carrier. An additional acid may be used that is characterized by vapor pressure >20 mmHg at 200° C., and the liquid nicotine formulation is heated by the electronic cigarette to generate an aerosol inhalable by the user.

Provided herein is a use of a liquid nicotine formulation for delivery of nicotine to a user from an electronic cigarette wherein the liquid nicotine formulation comprises a liquid nicotine formulation comprising nicotine and oxalic acid in a biologically acceptable liquid carrier. An additional acid may be used that is characterized by vapor pressure of about 20 to 200 mmHg at 200° C., and the liquid nicotine formulation is heated by the electronic cigarette to generate an aerosol inhalable by the user.

Provided herein is a use of a liquid nicotine formulation for delivery of nicotine to a user from an electronic cigarette wherein the liquid nicotine formulation comprises nicotine and oxalic acid in a biologically acceptable liquid carrier. An additional acid may be used that is characterized by a melting point <160° C., a boiling point >160° C., and at least a 50-degree difference between the melting point and the boiling point, and the liquid nicotine formulation is heated by the electronic cigarette to generate an aerosol inhalable by the user.

Provided herein is a use of a liquid nicotine formulation for delivery of nicotine to the blood of a user from an electronic cigarette, wherein the liquid nicotine formulation in the electronic cigarette is heated to form an aerosol which delivers a level of nicotine in the blood of the user that is at least 5 ng/mL at about 1.5 minutes after a first puff of ten puffs of the aerosol, each puff taken at 30 second intervals.

Provided herein is a use of a liquid nicotine formulation for delivery of nicotine to a user from an electronic cigarette wherein the liquid nicotine formulation comprises nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and the liquid nicotine formulation is heated by the electronic cigarette to generate an aerosol inhalable by the user.

Provided herein is a cartomizer for an electronic cigarette comprising:

a liquid nicotine formulation comprising nicotine and oxalic acid in a biologically acceptable liquid carrier. An additional acid may be used that is characterized by;

an atomizer comprising a heating element in fluid communication with the liquid nicotine formulation; and

a fluid storage compartment that stores the liquid nicotine formulation.

Provided herein is a cartomizer for an electronic cigarette comprising:

a liquid nicotine formulation comprising a liquid nicotine formulation comprising nicotine and oxalic acid in a biologically acceptable liquid carrier. An additional acid may be used that is characterized by vapor pressure of about 20 to 200 mmHg at 200° C.;

an atomizer comprising a heating element in fluid communication with the liquid nicotine formulation; and

a fluid storage compartment that stores the liquid nicotine formulation.

Provided herein is a cartomizer for an electronic cigarette comprising:

a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point;

an atomizer comprising a heating element in fluid communication with the liquid nicotine formulation; and

a fluid storage compartment that stores the liquid nicotine formulation.

Provided herein is a cartomizer for an electronic cigarette comprising:

a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point;

an atomizer comprising a heating element in fluid communication with the liquid nicotine formulation; and

a fluid storage compartment that stores the liquid nicotine formulation.

Provided herein is an electronic cigarette for generating an inhalable aerosol comprising:

a fluid storage compartment;

a heater; and

a liquid nicotine formulation in the fluid storage compartment, the liquid formulation comprising nicotine and oxalic acid in a biologically acceptable liquid carrier. An additional acid may be used that is characterized by vapor pressure >20 mmHg at 200° C.;

a battery; and

a mouthpiece.

Provided herein is an electronic cigarette for generating an inhalable aerosol comprising:

a fluid storage compartment;

a heater; and

a liquid nicotine formulation in the fluid storage compartment, the liquid formulation comprising nicotine and oxalic acid in a biologically acceptable liquid carrier. An additional acid may be used that is characterized by vapor pressure of about 20 to 200 mmHg at 200° C.;

a battery; and

a mouthpiece.

Provided herein is an electronic cigarette for generating an inhalable aerosol comprising:

a fluid storage compartment;

a heater; and

a liquid nicotine formulation in the fluid storage compartment, the liquid formulation comprising nicotine and oxalic acid in a biologically acceptable liquid carrier. An additional acid may be used that is characterized by a melting point <160° C., a boiling point >160° C., and at least a 50-degree difference between the melting point and the boiling point;

a battery; and

a mouthpiece.

Provided herein is an electronic cigarette for generating an inhalable aerosol comprising:

a fluid storage compartment;

a heater; and

a liquid nicotine formulation in the fluid storage compartment, the liquid formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point;

a battery; and

a mouthpiece.

Provided herein is a cartridge in an electronic cigarette comprising a fluid storage compartment, wherein the fluid storage compartment stores a liquid nicotine formulation comprising nicotine and oxalic acid in a biologically acceptable liquid carrier. An additional acid may be used that is characterized by vapor pressure >20 mmHg at 200° C.

Provided herein is a cartridge in an electronic cigarette comprising a fluid storage compartment, wherein the fluid storage compartment stores a liquid nicotine formulation comprising nicotine and oxalic acid in a biologically acceptable liquid carrier. An additional acid may be used that is characterized by vapor pressure of about 20 to 200 mmHg at 200° C.

Provided herein is a cartridge in an electronic cigarette comprising a fluid storage compartment, wherein the fluid storage compartment stores a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point.

Provided herein is a cartridge in an electronic cigarette comprising a fluid storage compartment, wherein the fluid storage compartment stores a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a kit comprising:

(a) an electronic cigarette for generating an inhalable aerosol comprising
i. a device body comprising a cartridge receptacle;
ii. a cartridge comprising a fluid storage compartment, wherein the fluid storage compartment stores a liquid nicotine formulation comprising nicotine and oxalic acid in a biologically acceptable liquid carrier. An additional acid may be used that is characterized by vapor pressure >20 mmHg at 200° C.;
iii. a heater;
iv. a battery; and
v. a mouthpiece; and

(b) instructions for using the electronic cigarette to generate an inhalable aerosol.

Provided herein is a kit comprising:

(c) an electronic cigarette for generating an inhalable aerosol comprising
i. a device body comprising a cartridge receptacle;
ii. a cartridge comprising a fluid storage compartment, wherein the fluid storage compartment stores a liquid nicotine formulation comprising nicotine and oxalic acid in a biologically acceptable liquid carrier. An additional acid may be used that is characterized by vapor pressure of about 20 to 200 mmHg at 200° C.;
iii. a heater;
iv. a battery; and
v. a mouthpiece; and

(d) instructions for using the electronic cigarette to generate an inhalable aerosol.

Provided herein is a kit comprising:

(e) an electronic cigarette for generating an inhalable aerosol comprising
i. a device body comprising a cartridge receptacle;
ii. a cartridge comprising a fluid storage compartment, wherein the fluid storage compartment stores a liquid nicotine formulation comprising nicotine and oxalic acid in a biologically acceptable liquid carrier. An additional acid may be used that is characterized by a melting point <160° C., a boiling point >160° C., and at least a 50-degree difference between the melting point and the boiling point;
iii. a heater;
iv. a battery; and
v. a mouthpiece; and

(f) instructions for using the electronic cigarette to generate an inhalable aerosol.

Provided herein is a kit comprising:

(g) an electronic cigarette for generating an inhalable aerosol comprising
i. a device body comprising a cartridge receptacle;
ii. a cartridge comprising a fluid storage compartment, wherein the fluid storage compartment stores a liquid nicotine formulation comprising nicotine and oxalic acid in a biologically acceptable liquid carrier. An additional acid may be used that is characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point;
iii. a heater;
iv. a battery; and
v. a mouthpiece; and

(h) instructions for using the electronic cigarette to generate an inhalable aerosol.

### Formulations

The formulations useful within the context of the present disclosure are described above in the context of liquid nicotine formulations. Provided here are further embodiments of these nicotine liquid formulations.

In some formulations, a dilute concentration of nicotine in the biologically compatible carrier is utilized. In some formulations, a less dilute concentration of nicotine in the biologically compatible carrier is utilized. In some formulations the concentration of nicotine in the liquid nicotine formulation is about 1% (w/w) to about 25% (w/w). In some formulations the concentration of nicotine in the liquid nicotine formulation is about 1% (w/w) to about 20% (w/w). In some formulations the concentration of nicotine in the liquid nicotine formulation is about 1% (w/w) to about 18% (w/w). In some embodiments the concentration of nicotine in the liquid nicotine formulation is about 1% (w/w) to about 15% (w/w). In some formulations the concentration of nicotine in the liquid nicotine formulation is about 4% (w/w) to about 12% (w/w). In some formulations the concentration of nicotine in the liquid nicotine formulation is about 4% (w/w). In some embodiments the concentration of nicotine in the liquid nicotine formulation is about 2% (w/w). In some formulations the concentration of nicotine in the liquid nicotine formulation is 1% (w/w) to 25% (w/w). In some formulations the concentration of nicotine in the liquid nicotine formulation is 1% (w/w) to 20% (w/w). In some formulations the concentration of nicotine in the liquid nicotine formulation is 1% (w/w) to 18% (w/w). In some formulations the concentration of nicotine in the liquid nicotine formulation is 1% (w/w) to 15% (w/w). In some formulations the concentration of nicotine in the liquid nicotine formulation is 4% (w/w) to 12% (w/w). In some formulations the concentration of nicotine in the liquid nicotine formulation is 4% (w/w). In some formulations the concentration of nicotine in the liquid nicotine formulation is 2% (w/w). As used with respect to concentrations of nicotine in the nicotine salt formulations, the term “about” refers to ranges of 0.05% (i.e. if the concentration is about 2%, the range is 1.95%-2.05%), 0.1 (i.e. if the concentration is about 2%, the range is 1.9%-2.1%), 0.25 (i.e. if the concentration is about 2%, the range is 1.75%-2.25%), 0.5 (i.e. if the concentration is about 2%, the range is 1.5%-2.5%), or 1 (i.e. if the concentration is about 4%, the range is 3%-5%), depending on the embodiment.

Where an additional acid is used as described above, nicotine salts are formed by the addition of a suitable acid, including organic or inorganic acids. In some formulations where an additional acid is used as described above, suitable organic acids are carboxylic acids. Examples of additional organic carboxylic acids disclosed herein are monocarboxylic acids, dicarboxylic acids (organic acid containing two carboxylic acid groups), carboxylic acids containing an aromatic group such as benzoic acids, hydroxycarboxylic acids, heterocyclic carboxylic acids, terpenoid acids, sugar acids; such as the pectic acids, amino acids, cycloaliphatic acids, aliphatic carboxylic acids, keto carboxylic acids, and the like. In some formulations provided herein, the organic acids used herein are monocarboxylic acids. Nicotine salts are formed from the addition of a suitable additional acid to nicotine

Nicotine is an alkaloid molecule that comprises two basic nitrogens. It may occur in different states of protonation. For example, if no protonation exists, nicotine is referred to as the “free base.” If one nitrogen is protonated, then the nicotine would be “mono-protonated.”

Liquid nicotine formulations may be formed by adding oxalic acid to nicotine, stirring the neat mixture at ambient temperature or at elevated temperature, and then diluting the neat mixture with a biologically compatible carrier mixture, such as a mixture of propylene glycol and glycerin. In some embodiments, the oxalic acid is completely dissolved by the nicotine prior to dilution. The oxalic acid may not completely dissolved by the nicotine prior to dilution. The addition of the oxalic acid to the nicotine to form a neat mixture may cause an exothermic reaction. The addition of the oxalic acid to the nicotine to form a neat mixture may be conducted at 55° C. The addition of the oxalic acid to the nicotine to form a neat mixture may be conducted at 90° C. The neat mixture may be cooled to ambient temperature prior to dilution. The dilution may be carried out at elevated temperature.

Additional nicotine salt formulations may be prepared by combining nicotine and a suitable additional acid in a biologically compatible carrier mixture, such as a mixture of propylene glycol and glycerin. The mixture of nicotine and a first biologically compatible carrier mixture is combined with a mixture of a suitable acid in a second biologically compatible carrier mixture. In some embodiments, the first and second biologically compatible carrier mixtures are identical in composition. In some embodiments, the first and second biologically compatible carrier mixtures are not identical in composition. In some embodiments, heating of nicotine/acid/biologically compatible carrier mixture is required to facilitate complete dissolution.

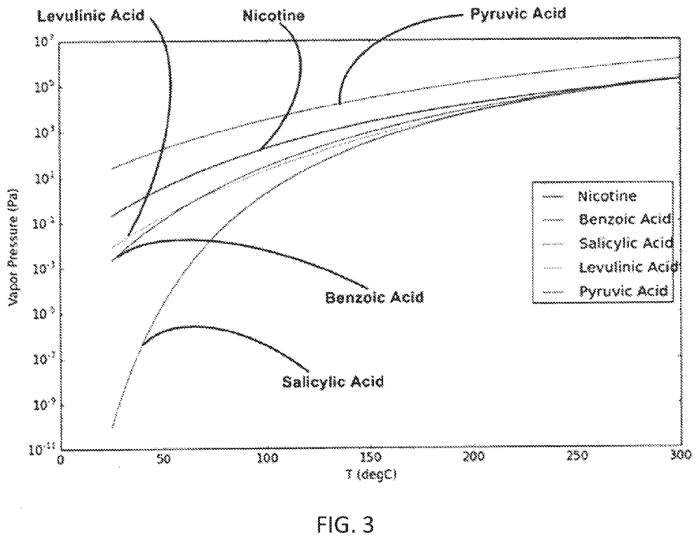

In some embodiments, liquid nicotine formulations may be prepared and added to a solution of 3:7 ratio by weight of propylene glycol (PG)/vegetable glycerin (VG), and mixed thoroughly. While described herein as producing 10 g of each of the formulations, all procedures noted infra are scalable. Other manners of formulation may also be employed form the formulations noted infra, without departing from the disclosure herein, and as would be known to one of skill in the art upon reading the disclosure herein.

Where an additional acid is utilized as described herein, the optimal liquid nicotine formulation may be determined by the vapor pressure of the additional acid. In some embodiments, the liquid nicotine formulations comprise an additional acid with a vapor pressure that is similar to the vapor pressure of free base nicotine. In some embodiments, the liquid nicotine formulations are formed from an additional acid with a vapor pressure that is similar to the vapor pressure of free base nicotine at the heating temperature of the device. FIG. 3 illustrates this trend. Nicotine salts formed from nicotine and benzoic acid; nicotine and salicylic acid; or nicotine and levulinic acid are salts that produce a satisfaction in an individual user consistent with efficient transfer of nicotine and a rapid rise in nicotine plasma levels. This pattern may be due to the mechanism of action during heating of the nicotine salt formulation. The nicotine salt may disassociate at, or just below, the heating temperature of the device, resulting in a mixture of free base nicotine and the individual acid. At that point, if both the nicotine and acid have similar vapor pressures, they may aerosolize at the same time, giving rise to a transfer of both free base nicotine and the constituent acid to the user.

The liquid nicotine formulation for generating an inhalable aerosol upon heating in an electronic cigarette may comprise an additional nicotine salt in a biologically acceptable liquid biologically compatible carrier; wherein the acid used to form the additional nicotine salt is characterized by a vapor pressure between 20-4000 mmHg at 200° C. In some embodiments, the acid used to form the additional nicotine salt is characterized by vapor pressure between 20-2000 mmHg at 200° C. In some embodiments, the acid used to form the additional nicotine salt is characterized by vapor pressure between 100-300 mmHg at 200° C.

The nicotine salt formed may be monoprotonated. The nicotine salt formed may be diprotonated. The nicotine salt may exist in more than one protonation state, e.g., an equilibrium of mono-protonated and di-protonated nicotine salts. The extent of protonation of the nicotine molecule may be dependent upon the stoichiometric ratio of nicotine to oxalic acid used in the liquid nicotine formulation. The extent of protonation of the nicotine molecule may be dependent upon the solvent. In some embodiments, monoprotonated nicotine produce a high degree of satisfaction in the user.

The flavor of the additional acid used in the liquid nicotine formulation may be a consideration in choosing the additional acid. A suitable additional acid may have minimal or no toxicity to humans in the concentrations used. A suitable additional acid may be compatible with the electronic cigarette components it contacts or could contact at the concentrations used. That is, such additional acid does not degrade or otherwise react with the electronic cigarette components it contacts or could contact. The odor of the additional acid used may be a consideration in choosing a suitable additional acid. The concentration of the additional nicotine salt in the biologically compatible carrier may affect the satisfaction in the individual user. In some embodiments, the flavor of the formulation is adjusted by changing the additional acid. In some embodiments, the flavor of the formulation is adjusted by adding exogenous flavorants. In some embodiments, an unpleasant tasting or smelling additional acid is used in minimal quantities to mitigate such characteristics. In some embodiments, exogenous pleasant smelling or tasting additional acid is added to the formulation. Examples of salts which can provide flavor and aroma to the mainstream aerosol at certain levels include nicotine acetate, nicotine oxalate, nicotine malate, nicotine isovalerate, nicotine lactate, nicotine citrate, nicotine phenylacetate and nicotine myristate.

Liquid nicotine formulations may generate an inhalable aerosol upon heating in an electronic cigarette. The molar amount of protonated nicotine inhaled may be user-determined. The user may, for example, by adjusting his inhalation strength.

The nicotine provided herein is either naturally occurring nicotine (e.g., from extract of nicotineous species such as tobacco), or synthetic nicotine. In some embodiments, the nicotine is (−)-nicotine, (+)-nicotine, or a mixture thereof In some embodiments, the nicotine is employed in relatively pure form (e.g., greater than about 80% pure, 85% pure, 90% pure, 95% pure, or 99% pure). In some embodiments, the nicotine provided herein is “water clear” in appearance in order to avoid or minimize the formation of tarry residues during the subsequent salt formation steps.

The formulation further may comprise one or more flavorants.

The suitable additional acid for the liquid nicotine formulation may have a vapor pressure >20 mmHg at 200° C. and is non-corrosive to the electronic cigarette or is non-toxic to humans. In some embodiments, the suitable additional acid for the liquid nicotine formulation is selected from the group consisting of salicylic acid, formic acid, sorbic acid, acetic acid, benzoic acid, pyruvic acid, lauric acid, and levulinic acid.

The suitable additional acid for the nicotine salt formulation may have a vapor pressure of about 20 to 200 mmHg at 200° C. and is non-corrosive to the electronic cigarette or is non-toxic to humans. In some embodiments, the suitable additional acid for the liquid nicotine formulation s selected from the group consisting of salicylic acid, benzoic acid, lauric acid, and levulinic acid.

The suitable additional acid for the liquid nicotine formulation may have a melting point <160° C., a boiling point >160° C., at least a 50-degree difference between the melting point and the boiling point, and is non-corrosive to the electronic cigarette or is non-toxic to humans. In some embodiments, the suitable additional acid for the liquid nicotine formulation has a melting point at least 40 degrees lower than the operating temperature of the electronic cigarette, a boiling point no more than 40 degrees lower than the operating temperature of the electronic cigarette, at least a 50-degree difference between the melting point and the boiling point, and is non-corrosive to the electronic cigarette or is non-toxic to humans; wherein the operating temperature is 200° C. In some embodiments, the suitable additional acid for the liquid nicotine formulation is selected from the group consisting of salicylic acid, sorbic acid, benzoic acid, pyruvic acid, lauric acid, and levulinic acid.

The suitable additional acid for the liquid nicotine formulation does not decompose at the operating temperature of the electronic cigarette. In some embodiments, the suitable additional acid for the liquid nicotine formulation does not oxidize at the operating temperature of the electronic cigarette. In some embodiments, the suitable additional acid for the additional nicotine salt formation does not oxidize at room temperature. In some embodiments, the suitable additional acid for the liquid nicotine formulation does not provide an unpleasant taste. In some embodiments, the suitable additional acid for the liquid nicotine formulation has good solubility in a liquid formulation for use in an electronic cigarette.

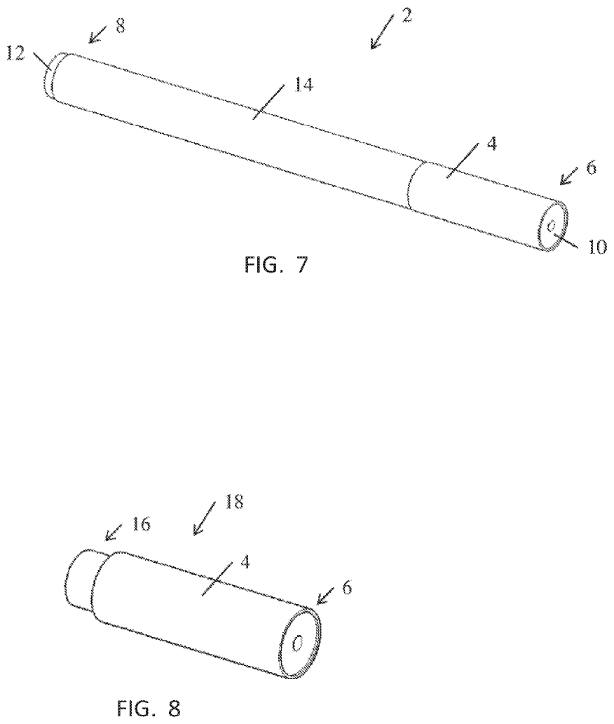

Provided herein is an electronic cigarette 2 having a fluid storage compartment 4 comprising an embodiment liquid nicotine formulation of any embodiment described herein within the fluid storage compartment described herein. An embodiment is shown in FIG. 7. The electronic cigarette 2 of FIG. 7 includes a mouth end 6, and a charging end 8. The mouth-end 6 includes a mouthpiece 10. The charging end 8 may connect to a battery or a charger or both, wherein the battery is within a body of the electronic cigarette, and the charger is separate from the battery and couples to the body or the battery to charge the battery. In some embodiments the electronic cigarette comprises a rechargeable battery within a body 14 of the electronic cigarette and the charge end 8 comprises a connection 12 for charging the rechargeable battery. In some embodiments, the electronic cigarette comprises a cartomizer that comprises the fluid storage compartment and an atomizer. In some embodiments, the atomizer comprises a heater. In some embodiments the fluid storage compartment 4 is separable from an atomizer. In some embodiments the fluid storage compartment 4 is replaceable as part of a replaceable cartridge. In some embodiments the fluid storage compartment 4 is refillable. In some embodiments, the mouthpiece 10 is replaceable.

Provided herein is a cartomizer 18 for an electronic cigarette 2 having a fluid storage compartment 4 comprising an embodiment liquid nicotine formulation of any embodiment described herein within the fluid storage compartment described herein. The cartomizer 18 embodiment of FIG. 8 includes a mouth end 6, and a connection end 16. The connection end 16 in the embodiment of FIG. 8 couples the cartomizer 14 to a body of an electronic cigarette, or to a battery of the electronic cigarette, or both. The mouth end 6 includes a mouthpiece 10. In some embodiments, the cartomizer does not include a mouthpiece, and in such embodiments, the cartomizer can be coupled to a mouthpiece of an electronic cigarette, or the cartomizer can be coupled to a battery or body of an electronic cigarette, while the mouthpiece is also coupled to the battery or the body of the electronic cigarette. In some embodiments, the mouthpiece is integral with the body of the electronic cigarette. In some embodiments, including the embodiment of FIG. 8, the cartomizer 18 comprises the fluid storage compartment 4 and an atomizer (not shown). In some embodiments, the atomizer comprises a heater (not shown).

### EMBODIMENTS

Embodiment 1. A method of delivering protonated nicotine to a user of an electronic cigarette, the method including: (a) operating an electronic cigarette including a liquid nicotine formulation, the liquid nicotine including nicotine, oxalic acid and a biologically acceptable liquid carrier; (b) heating the liquid nicotine formulation to an operating temperature, wherein the heating provides an inhalable aerosol including an effective amount of protonated nicotine; and (c) inhaling the inhalable aerosol.

Embodiment 2. The method of embodiment 1, wherein the oxalic acid and the nicotine form a nicotine salt.

Embodiment 3. The method of embodiments 1 or 2, wherein the molar ratio of nicotine to oxalic acid functional groups is about 1:2 or greater.

Embodiment 4. The method of any of embodiments 1 to 3, wherein the molar ratio of nicotine to oxalic acid is from about 1:0.5 to about 1:1.5.

Embodiment 5. The method of any one of embodiments 1 to 4, wherein the molar ratio of nicotine to oxalic acid is about 1:1.

Embodiment 6. The method of any one of embodiments 1 to 5, wherein the number of oxalic acid functional groups is equal to or greater than the number of nicotine molecules.

Embodiment 7. The method of any one of embodiments 1 to 6, wherein the moles of oxalic acid functional groups is greater than the moles of nicotine.

Embodiment 8. The method of any one of embodiments 1 to 7, wherein the moles of oxalic acid functional groups is from about 1.1 times greater to about 3.0 times greater than the moles of nicotine.

Embodiment 9. The method of any one of embodiments 1 to 8, wherein the moles of oxalic acid functional groups is from about 1.5 times greater to about 2.2 times greater than the moles of nicotine.

Embodiment 10. The method of any one of embodiments 1 to 9, wherein the liquid nicotine formulation includes about 4% to about 5% nicotine (w/w) and about 2.5% to about 3.5% oxalic acid (w/w).

Embodiment 11. The method of any one of embodiments 1 to 10, wherein the liquid nicotine formulation includes about 5% nicotine (w/w) and about 2.8% (w/w) oxalic acid.

Embodiment 12. The method of any one of embodiments 1 to 11, wherein the nicotine salt is in an amount that forms about 1% to about 20% nicotine in the inhalable aerosol.

Embodiment 13. The method of any one of embodiments 1 to 12, wherein the liquid nicotine formulation has a nicotine concentration of from about 0.5% (w/w) to about 20% (w/w).

Embodiment 14. The method of any one of embodiments 1 to 13, wherein the operating temperature is from about150° C. to about 250° C.

Embodiment 15. The method of any one of embodiments 1 to14, wherein the operating temperature is from about 180° C. to about 220° C.

Embodiment 16. The method of any one of embodiments 1 to 15, wherein the operating temperature is 200° C.

Embodiment 17. The method of any one of embodiments 1 to 16, wherein the aerosol includes a condensate of the nicotine salt.

Embodiment 18. The method of any one of embodiments 1-17, wherein the nicotine salt is nicotine oxalate.

Embodiment 19. The method any one of embodiments 1-18, wherein the aerosol includes condensate in particle sizes from about 0.1 microns to about 5 microns.

Embodiment 20. The method any one of embodiments 1-19, wherein the aerosol includes condensate in particle sizes from about from about 0.1 microns to about 1 or 2 microns.

Embodiment 21. The method any one of embodiments 1-20, wherein the aerosol includes condensate in particle sizes from about from about 0.1 microns to about 0.7 microns.

Embodiment 22. The method any one of embodiments 1-21, wherein the aerosol includes condensate in particle sizes from about from about 0.3 microns to about 0.4 microns.

Embodiment 23. The method of any one of embodiments 1-22, wherein the liquid carrier includes glycerol, propylene glycol, trimethylene glycol, water, ethanol or combinations thereof.

Embodiment 24. The method of any one of embodiments 1-23, wherein the liquid carrier includes propylene glycol and vegetable glycerin.

Embodiment 25. The method of any one of embodiments 1-24, wherein the liquid carrier includes about 20% to about 50% of propylene glycol and about 80% to about 50% of vegetable glycerin.

Embodiment 26. The method of any one of embodiments 1-25, wherein the liquid carrier includes about 30% propylene glycol and about 70% vegetable glycerin.

Embodiment 27. The method of any one of embodiments 1-26, wherein the nicotine salt is in an amount that forms about 0.5% to about 20% nicotine in the inhalable aerosol or about 1% to about 20% nicotine in the inhalable aerosol.

Embodiment 28. The method of any one of embodiments 1-27, wherein the formulation further includes one or more additional nicotine salts in a biologically acceptable liquid carrier suitable for generating the inhalable aerosol upon heating.

Embodiment 29. The method of embodiment 28, wherein a second acid is used to form the additional nicotine salt.

Embodiment 30. The method of embodiment 29, wherein the second acid is selected from the group consisting of salicylic acid, sorbic acid, benzoic acid, pyruvic acid, lauric acid, and levulinic acid.

Embodiment 31. The method of any one of embodiments 1-30, wherein the formulation further includes a flavorant.

Embodiment 32. The method of any one of embodiments 1-31, wherein the effective amount results in the user experiencing less respiratory tract harshness relative to a control liquid nicotine formulation including an equivalent amount of an acid other than oxalic acid.

Embodiment 33. The method of embodiment any one of embodiments 1-32,wherein the second acid is selected from the group consisting of levulinic acid, benzoic acid, succinic acid, salicylic acid, malic acid, pyruvic acid, citric acid, lauric acid and sorbic acid.

Embodiment 34. A liquid nicotine formulation including nicotine, oxalic acid and a biologically acceptable liquid carrier, wherein upon heating the liquid nicotine formulation an inhalable aerosol is formed including an effective amount of protonated nicotine.

Embodiment 35. The liquid nicotine formulation of embodiment 34, wherein the liquid nicotine formulation is in a cartridge.

Embodiment 36. The liquid nicotine formulation of embodiment 35, wherein the cartridge is in an electronic cigarette.

Embodiment 37. The liquid nicotine formulation of embodiment 34, wherein the oxalic acid and the nicotine form a nicotine salt.

Embodiment 38. The liquid nicotine formulation of embodiments 34 or 37, wherein the molar ratio of nicotine to oxalic acid functional groups is about 1:2 or greater.

Embodiment 39. The liquid nicotine formulation of any of embodiments 34 to 38, wherein the molar ratio of nicotine to oxalic acid is from about 1:0.5 to about 1:1.5.

Embodiment 40. The liquid nicotine formulation of any one of embodiments 34 to 39, wherein the molar ratio of nicotine to oxalic acid is about 1:1.

Embodiment 41. The liquid nicotine formulation of any one of embodiments 34 to 40, wherein the number of oxalic acid functional groups is equal to or greater than the molar amount of nicotine.

Embodiment 42. The liquid nicotine formulation of any one of embodiments 34 to 41, wherein the moles of oxalic acid functional groups is equal to or greater than the moles of nicotine.

Embodiment 43. The liquid nicotine formulation of any one of embodiments 34 to 42, wherein the moles of oxalic acid functional groups is from about 1.1 times greater to about 3.0 times greater than the moles of nicotine.

Embodiment 44. The liquid nicotine formulation of any one of embodiments 34 to 43, wherein the moiles of oxalic acid functional groups is from about 1.5 times greater to about 2.2 times greater than the molar amount of nicotine.

Embodiment 45. The liquid nicotine formulation of any one of embodiments 34 to 44, wherein the liquid nicotine formulation includes about 4% to about 5% nicotine (w/w) and about 2.5% to about 3.5% oxalic acid (w/w).

Embodiment 46. The liquid nicotine formulation of any one of embodiments 34 to 45, wherein the liquid nicotine formulation includes about 5% nicotine (w/w) and about 2.8% (w/w) oxalic acid.

Embodiment 47. The liquid nicotine formulation of any one of embodiments 34 to 46, wherein the nicotine salt is in an amount that forms about 1% to about 20% nicotine in the inhalable aerosol.

Embodiment 48. The liquid nicotine formulation of any one of embodiments 34 to 47, wherein the liquid nicotine formulation has a nicotine concentration of from about 0.5% (w/w) to about 20% (w/w).

Embodiment 49. The liquid nicotine formulation of any one of embodiments 34 to 48, wherein the operating temperature is from about150° C. to about 250° C.

Embodiment 50. The liquid nicotine formulation of any one of embodiments 34 to 49, wherein the operating temperature is from about 180° C. to about 220° C.

Embodiment 51. The liquid nicotine formulation of any one of embodiments 34 to 50, wherein the operating temperature is 200° C.

Embodiment 52. The liquid nicotine formulation of any one of embodiments 34 to 51, wherein the aerosol includes a condensate of the nicotine salt.

Embodiment 53. The liquid nicotine formulation of any one of embodiments 34 to 52, wherein the nicotine salt is nicotine oxalate.

Embodiment 54. The liquid nicotine formulation any one of embodiments 34 to 53, wherein the aerosol includes condensate in particle sizes from about 0.1 microns to about 5 microns.

Embodiment 55. The liquid nicotine formulation any one of embodiments 34 to 54, wherein the aerosol includes condensate in particle sizes from about from about 0.1 microns to about 1 or 2 microns.

Embodiment 56. The liquid nicotine formulation any one of embodiments 34 to 55, wherein the aerosol includes condensate in particle sizes from about from about 0.1 microns to about 0.7 microns.

Embodiment 57. The liquid nicotine formulation any one of embodiments 34 to 56, wherein the aerosol includes condensate in particle sizes from about from about 0.3 microns to about 0.4 microns.

Embodiment 58. The liquid nicotine formulation of any one of embodiments 34 to 57, wherein the liquid carrier includes glycerol, propylene glycol, trimethylene glycol, water, ethanol or combinations thereof.

Embodiment 59. The liquid nicotine formulation of any one of embodiments 34 to 58, wherein the liquid carrier includes propylene glycol and vegetable glycerin.

Embodiment 60. The liquid nicotine formulation of any one of embodiments 34 to 59, wherein the liquid carrier includes about 20% to about 50% of propylene glycol and about 80% to about 50% of vegetable glycerin.

Embodiment 61. The liquid nicotine formulation of any one of embodiments 34 to 60, wherein the liquid carrier includes about 30% propylene glycol and about 70% vegetable glycerin.

Embodiment 62. The liquid nicotine formulation of any one of embodiments 34 to 61, wherein the nicotine salt is in an amount that forms about 0.5% to about 20% nicotine in the inhalable aerosol or about 1% to about 20% nicotine in the inhalable aerosol.

Embodiment 63. The liquid nicotine formulation of any one of embodiments 34 to 62, wherein the formulation further includes one or more additional nicotine salts in a biologically acceptable liquid carrier suitable for generating the inhalable aerosol upon heating.

Embodiment 64. The liquid nicotine formulation of embodiment 63, wherein an additional acid is used to form the additional nicotine salt.

Embodiment 65. The liquid nicotine formulation of embodiment 64, wherein the additional acid is selected from the group consisting of salicylic acid, sorbic acid, benzoic acid, pyruvic acid, lauric acid, and levulinic acid.

Embodiment 66. The liquid nicotine formulation of any one of embodiments 34 to 64, wherein the formulation further includes a flavorant.

Embodiment 67. The liquid nicotine formulation of any one of embodiments 34 to 65, wherein the effective amount results in a user inhaling the aerosol experiencing less respiratory tract harshness relative to a control liquid nicotine formulation including an equivalent amount of an acid other than oxalic acid.

Embodiment 68. The liquid formulation of embodiment 67, wherein the acid other than oxalic acid is selected from the group consisting of levulinic acid, benzoic acid, succinic acid, salicylic acid, malic acid, pyruvic acid, citric acid, lauric acid and sorbic acid.

### OTHER EMBODIMENTS

Embodiment P 1. A method of delivering nicotine to a user comprising: wicking a nicotine salt solution from a liquid reservoir into a resistive heater, wherein the nicotine salt solution comprises nicotine, oxalic acid, and a biologically acceptable liquid biologically compatible carrier, wherein the nicotine is protonated and wherein the ratio of nicotine to acid functional groups is 1:2 or greater; and applying current to vaporizer the nicotine solution wicked into thermal contact with the resistive heater to form an inhalable aerosol, further wherein the nicotine concentration is from about 0.5% (w/w) to about 20% (w/w).

### EXAMPLES

### Example 1: Preparation of Nicotine Salt Formulations

Various nicotine formulations were prepared and added to a solution of 3:7 ratio by weight of propylene glycol (PG)/vegetable glycerin (VG), and mixed thoroughly. The examples shown below were used to make lOg of each of the formulations. All procedures are scalable.

For example, in order to make nicotine formulations with a final nicotine free base equivalent concentration of 2% (w/w), the following procedures were applied to each individual formulation.
Nicotine benzoate salt formulation: 0.15 g benzoic acid was added to a beaker followed by adding 0.2 g nicotine to the same beaker. The mixture was stirred at 55° C. for 20 minutes until benzoic acid was completely dissolved and an orange oily mixture was formed. The mixture was cooled down to ambient conditions. 9.65 g PG/VG (3:7) solution was added to the orange nicotine benzoate salt and the mixture was stirred until a visually homogenous formulation solution was achieved.
Nicotine benzoate salt formulation can also be made by adding 0.15 g benzoic acid to a beaker followed by adding 0.2 g nicotine and 9.65 g PG/VG (3:7) solution to the same beaker. The mixture was then stirred at 55° C. for 20 minutes until a visually homogenous formulation solution was achieved with no undissolved chemicals.
Nicotine citrate salt formulation was made by adding 0.47 g citric acid to a beaker followed by adding 0.2 g nicotine and 9.33 g PG/VG (3:7) solution to the same beaker. The mixture was then stirred at 90° C. for 60 minutes until a visually homogenous formulation solution was achieved with no undissolved chemicals.
Nicotine malate salt formulation was made by adding 0.33 g L-malic acid to a beaker followed by adding 0.2 g nicotine and 9.47 g PG/VG (3:7) solution to the same beaker. The mixture was then stirred at 90° C. for 60 minutes until a visually homogenous formulation solution was achieved with no undissolved chemicals.
Nicotine succinate salt formulation was made by adding 0.29 g succinic acid to a beaker followed by adding 0.2 g nicotine and 9.51 g PG/VG (3:7) solution to the same beaker. The mixture was then stirred at 90° C. for 60 minutes until a visually homogenous formulation solution was achieved with no undissolved chemicals.
Nicotine salicylate salt formulation was made by adding 0.17 g salicylic acid to a beaker followed by adding 0.2 g nicotine and 9.63 g PG/VG (3:7) solution to the same beaker. The mixture was then stirred at 90° C. for 60 minutes until a visually homogenous formulation solution was achieved with no undissolved chemicals.
Nicotine salicylate salt formulation can also be made by adding 0.17 g salicylic acid to a beaker followed by adding 0.2 g nicotine to the same beaker. The mixture was stirred at 90° C. for 60 minutes until salicylic acid was completely dissolved and an orange oily mixture was formed. The mixture was either cooled to ambient conditions or kept at 90° C. when 9.63 g PG/VG (3:7) solution was added. The mixture was then stirred at 90° C. until a visually homogenous formulation solution was achieved with no undissolved chemicals.
Nicotine free base formulation was made by adding 0.2 g nicotine to a beaker followed by adding 9.8 g PG/VG (3:7) solution to the same beaker. The mixture was then stirred at ambient conditions for 10 minutes until a visually homogenous formulation solution was achieved.

For example, in order to make nicotine salt formulations with a final nicotine free base equivalent concentration of 3% (w/w), the following procedures were applied to each individual formulation.
Nicotine benzoate salt formulation: 0.23 g benzoic acid was added to a beaker followed by adding 0.3 g nicotine to the same beaker. The mixture was stirred at 55° C. for 20 minutes until benzoic acid was completely dissolved and an orange oily mixture was formed. The mixture was cooled down to ambient conditions. 9.47 g PG/VG (3:7) solution was added to the orange nicotine benzoate salt and the blend was stirred until a visually homogenous formulation solution was achieved.
Nicotine benzoate salt formulation can also be made by adding 0.23 g benzoic acid to a beaker followed by adding 0.3 g nicotine and 9.47 g PG/VG (3:7) solution to the same beaker. The mixture was then stirred at 55° C. for 20 minutes until a visually homogenous formulation solution was achieved with no undissolved chemicals.
Nicotine citrate salt formulation was made by adding 0.71 g citric acid to a beaker followed by adding 0.3 g nicotine and 8.99 g PG/VG (3:7) solution to the same beaker. The mixture was then stirred at 90° C. for 60 minutes until a visually homogenous formulation solution was achieved with no undissolved chemicals.
Nicotine malate salt formulation was made by adding 0.5 g L-malic acid to a beaker followed by adding 0.3 g nicotine and 9.2 g PG/VG (3:7) solution to the same beaker. The mixture was then stirred at 90° C. for 60 minutes until a visually homogenous formulation solution was achieved with no undissolved chemicals.
Nicotine levulinate salt formulation was made by adding melted 0.64 g levulinic acid to a beaker followed by adding 0.3 g nicotine to the same beaker. The mixture was stirred at ambient conditions for 10 minutes. Exothermic reaction took place and oily product was produced. The mixture was allowed to cool down to ambient temperature and 9.06 g PG/VG (3:7) solution was added to the same beaker. The mixture was then stirred at ambient conditions for 20 minutes until a visually homogenous formulation solution was achieved.
Nicotine pyruvate salt formulation was made by adding 0.33 g pyruvic acid to a beaker followed by adding 0.3 g nicotine to the same beaker. The mixture was stirred at ambient conditions for 10 minutes. Exothermic reaction took place and oily product was produced. The mixture was allowed to cool down to ambient temperature and 9.37 g PG/VG (3:7) solution was added to the same beaker. The mixture was then stirred at ambient conditions for 20 minutes until a visually homogenous formulation solution was achieved.
Nicotine succinate salt formulation was made by adding 0.44 g succinic acid to a beaker followed by adding 0.3 g nicotine and 9.26 g PG/VG (3:7) solution to the same beaker. The mixture was then stirred at 90° C. for 60 minutes until a visually homogenous formulation solution was achieved with no undissolved chemicals.
Nicotine salicylate salt formulation was made by adding 0.26 g salicylic acid to a beaker followed by adding 0.3 g nicotine and 9.44 g PG/VG (3:7) solution to the same beaker. The mixture was then stirred at 90° C. for 60 minutes until a visually homogenous formulation solution was achieved with no undissolved chemicals.
Nicotine salicylate salt formulation can also be made by adding 0.26 g salicylic acid to a beaker followed by adding 0.3 g nicotine to the same beaker. The mixture was stirred at 90° C. for 60 minutes until salicylic acid was completely dissolved and an orange oily mixture was formed. The mixture was either cooled to ambient conditions or kept at 90° C. when 9.44 g PG/VG (3:7) solution was added. The blend was then stirred at 90 C until a visually homogenous formulation solution was achieved with no undissolved chemicals.
Nicotine free base formulation was made by adding 0.3 g nicotine to a beaker followed by adding 9.7 g PG/VG (3:7) solution to the same beaker. The mixture was then stirred at ambient conditions for 10 minutes until a visually homogenous formulation solution was achieved.

For example, in order to make nicotine salt formulations with a final nicotine free base equivalent concentration of 4% (w/w), the following procedures were applied to each individual formulation.
Nicotine benzoate salt formulation: 0.3 g benzoic acid was added to a beaker followed by adding 0.4 g nicotine to the same beaker. The mixture was stirred at 55° C. for 20 minutes until benzoic acid was completely dissolved and an orange oily mixture was formed. The mixture was cooled down to ambient conditions. 9.7 g PG/VG (3:7) solution was added to the orange nicotine benzoate salt and the blend was stirred until a visually homogenous formulation solution was achieved.
Nicotine benzoate salt formulation can also be made by adding 0.3 g benzoic acid to a beaker followed by adding 0.4 g nicotine and 9.7 g PG/VG (3:7) solution to the same beaker. The mixture was then stirred at 55° C. for 20 minutes until a visually homogenous formulation solution was achieved with no undissolved chemicals.

For example, in order to make nicotine salt formulations with a final nicotine free base equivalent concentration of 5% (w/w), the following procedures were applied to each individual formulation.
Nicotine benzoate salt formulation: 0.38 g benzoic acid was added to a beaker followed by adding 0.5 g nicotine to the same beaker. The mixture was stirred at 55° C. for 20 minutes until benzoic acid was completely dissolved and an orange oily mixture was formed. The mixture was cooled down to ambient conditions. 9.12 g PG/VG (3:7) solution was added to the orange nicotine benzoate salt and the blend was stirred until a visually homogenous formulation solution was achieved.
Nicotine benzoate salt formulation can also be made by adding 0.38 g benzoic acid to a beaker followed by adding 0.5 g nicotine and 9.12 g PG/VG (3:7) solution to the same beaker. The mixture was then stirred at 55° C. for 20 minutes until a visually homogenous formulation solution was achieved with no undissolved chemicals.
Nicotine malate salt formulation was made by adding 0.83 g L-malic acid to a beaker followed by adding 0.5 g nicotine and 8.67 g PG/VG (3:7) solution to the same beaker. The mixture was then stirred at 90° C. for 60 minutes until a visually homogenous formulation solution was achieved with no undissolved chemicals.
Nicotine levulinate salt formulation was made by adding melted 1.07 g levulinic acid to a beaker followed by adding 0.5 g nicotine to the same beaker. The mixture was stirred at ambient conditions for 10 minutes. Exothermic reaction took place and oily product was produced. The mixture was allowed to cool down to ambient temperature and 8.43 g PG/VG (3:7) solution was added to the same beaker. The mixture was then stirred at ambient conditions for 20 minutes until a visually homogenous formulation solution was achieved.
Nicotine pyruvate salt formulation was made by adding 0.54 g pyruvic acid to a beaker followed by adding 0.5 g nicotine to the same beaker. The mixture was stirred at ambient conditions for 10 minutes. Exothermic reaction took place and oily product was produced. The mixture was allowed to cool down to ambient temperature and 8.96 g PG/VG (3:7) solution was added to the same beaker. The mixture was then stirred at ambient conditions for 20 minutes until a visually homogenous formulation solution was achieved.
Nicotine succinate salt formulation was made by adding 0.73 g succinic acid to a beaker followed by adding 0.5 g nicotine and 8.77 g PG/VG (3:7) solution to the same beaker. The mixture was then stirred at 90° C. for 60 minutes until a visually homogenous formulation solution was achieved with no undissolved chemicals.
Nicotine salicylate salt formulation was made by adding 0.43 g salicylic acid to a beaker followed by adding 0.5 g nicotine and 9.07 g PG/VG (3:7) solution to the same beaker. The mixture was then stirred at 90° C. for 60 minutes until a visually homogenous formulation solution was achieved with no undissolved chemicals.
Nicotine salicylate salt formulation can also be made by adding 0.43 g salicylic acid to a beaker followed by adding 0.5 g nicotine to the same beaker. The mixture was stirred at 90° C. for 60 minutes until salicylic acid was completely dissolved and an orange oily mixture was formed. The mixture was either cooled to ambient conditions or kept at 90 C when 9.07 g PG/VG (3:7) solution was added. The blend was then stirred at 90° C. until a visually homogenous formulation solution was achieved with no undissolved chemicals.
Nicotine free base formulation was made by adding 0.5 g nicotine to a beaker followed by adding 9.5 g PG/VG (3:7) solution to the same beaker. The mixture was then stirred at ambient conditions for 10 minutes until a visually homogenous formulation solution was achieved.

Various formulations comprising different nicotine salts can be prepared similarly, or different concentrations of the above-noted nicotine formulations or other nicotine salt formulations can be prepared as one of skill in the art would know to do upon reading the disclosure herein.

Various formulations comprising two or more nicotine salts can be prepared similarly in a solution of 3:7 ratio of propylene glycol (PG)/vegetable glycerin (VG). For example, 0.43 g (2.5% w/w nicotine) of nicotine levulinate salt and 0.34 g (2.5% w/w nicotine) of nicotine acetate salt are added to 9.23 g of PG/VG solution, to achieve a 5% w/w nicotine formulation.

Also provided is another exemplary formulation. For example, 0.23 g (1.33% w/w nicotine) of nicotine benzoate salt (molar ratio 1:1 nicotine/benzoic acid), 0.25 g (1.33% w/w nicotine) of nicotine salicylate salt(molar ratio 1:1 nicotine/salicylic acid) and 0.28 g (1.34% w/w nicotine) of nicotine pyruvate salt (molar ratio 1:2 nicotine/pyruvic acid) are added to 9.25 g of PG/VG solution, to achieve a 5% w/w nicotine formulation.

### Example 2: Heart Rate Study of Nicotine Solutions Via E-Cigarette

Exemplary formulations of nicotine levulinate, nicotine benzoate, nicotine succinate, nicotine salicylate, nicotine malate, nicotine pyruvate, nicotine citrate, nicotine freebase, and a control of propylene glycol were prepared as noted in Example 1 in 3% w/w solutions and were administered in the same fashion by an electronic cigarette to the same human subject. About 0.5 mL of each solution was loaded into an “eRoll” cartridge atomizer (joyetech.com) to be used in the study. The atomizer was then attached to an “eRoll” e-cigarette (same manufacturer). The operating temperature was from about 150° C. to about 250° C., or from about 180° C. to about 220° C.

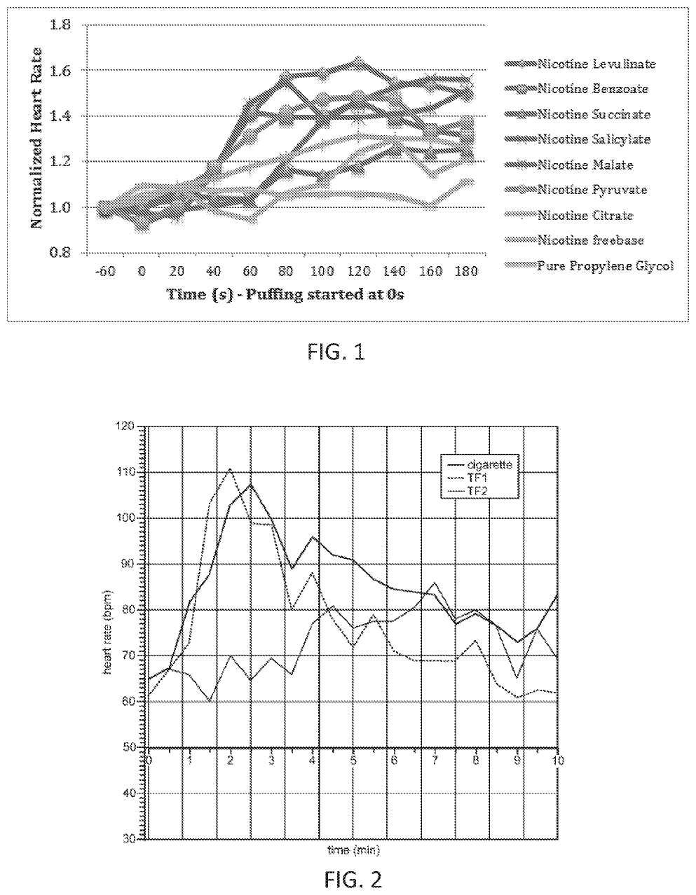

Heart rate measurements were taken for 6 minutes; from 1 minute before start of puffing, for 3 minutes during puffing, and continuing until 2 minutes after end of puffing. The test participant took 10 puffs over 3 minutes in each case. The base heart rate was the average heart rate over the first 1 minute before start of puffing. Heart rate after puffing started was averaged over 20-second intervals. Puffing (inhalation) occurred every 20 seconds for a total of 3 minutes. Normalized heart rate was defined as the ratio between individual heart rate data point and the base heart rate. Final results were presented as normalized heart rate, shown for the first 4 minutes in FIG. 1.

FIG. 1 summarizes results from heart rate measurements taken for a variety of nicotine salt formulations. For ease of reference in reviewing FIG. 1, at the 180-second timepoint, from top to bottom (highest normalized heart rate to lowest normalized heart rate), the nicotine formulations are as follows: nicotine salicylate formulation, nicotine malate formulation, nicotine levulinate formulation (nearly identical to nicotine malate formulation at 180 seconds, thus, as a second reference point: the nicotine malate formulation curve is lower than the nicotine levulinate formulation curve at the 160-second time point), nicotine pyruvate formulation, nicotine benzoate formulation, nicotine citrate formulation, nicotine succinate formulation, and nicotine free base formulation. The bottom curve (lowest normalized heart rate) at the 180-second timepoint is associated with the placebo (100% propylene glycol). The test formulations comprising a nicotine salt cause a faster and more significant rise in heart rate than the placebo. The test formulations comprising a nicotine salt also cause faster and more significant rise when compared with a nicotine freebase formulation with the same amount of nicotine by weight. In addition, the nicotine salts (e.g., nicotine benzoate and nicotine pyruvate) prepared from the acids having calculated vapor pressures between 20-200 mmHg at 200° C. (benzoic acid (171.66 mmHg), with the exception of pyruvic acid (having a boiling point of 165 C), respectively) cause a faster rise in heart rate than the rest. The nicotine salts (e.g., nicotine levulinate, nicotine benzoate, and nicotine salicylate) prepared from the acids (benzoic acid, levulinic acid and salicylic acid, respectively) also cause a more significant heart rate increase. Thus, other suitable nicotine salts formed by the acids with the similar vapor pressure and/or similar boiling point may be used in accordance with the practice of the present embodiments and aspects. This experience of increased heart rate theoretically approaching or theoretically comparable to that of a traditional burned cigarette has not been demonstrated or identified in other electronic cigarette devices. Nor has it been demonstrated or identified in low temperature tobacco vaporization devices (electronic cigarettes) that do not burn the tobacco, even when a nicotine salt was used (a solution of 20% (w/w) or more of nicotine salt) as an additive to the tobacco. Thus the results from this experiment are surprising and unexpected.

### Example 3: Satisfaction Study of Nicotine Salt Solution Via E-Cigarette

In addition to the heart rate study shown in Example 2, nicotine formulations (using 3% w/w nicotine formulations as described in Example 1) were used to conduct a satisfaction study in a single test participant. The test participant, an e-cigarette and/or traditional cigarette user, was required to have no nicotine intake for at least 12 hours before the test. The participant took 10 puffs using an e-cigarette (same as used in Example 2) over 3 minutes in each case, and then was asked to rate the level of physical and emotional satisfaction he or she felt on a scale of 0-10, with 0 being no physical or emotional satisfaction. The results indicated that the least satisfying compound was the nicotine free base. Nicotine benzoate, nicotine salicylate, and nicotine succinate all performed well, followed by nicotine pyruvate, nicotine citrate, and nicotine pyruvate.

Based on the Satisfaction Study, the nicotine salts formulations with acids having vapor pressure ranges between >20 mmHg @ 200° C., or 20-200 mmHg @ 200° C., or 100-300 mmHg @ 200° C. provide more satisfaction than the rest (except the pyruvic acid which has boiling point of 165° C.). For reference, it has been determined that salicylic acid has a vapor pressure of about 135.7 mmHg @ 200° C., benzoic acid has a vapor pressure of about 171.7 mmHg @ 200° C., lauric acid has a vapor pressure of about 38 mmHg @ 200° C., and levulinic acid has a vapor pressure of about 149 mmHg @ 200° C.

### Example 4: Test Formulation 1 (TF1)

A solution of nicotine levulinate in glycerol comprising nicotine salt used: 1.26 g (12.6% w/w) of 1:3 nicotine levulinate 8.74 g (87.4% w/w) of glycerol—Total weight 10.0 g.

Neat nicotine levulinate was added to the glycerol, and mixed thoroughly. L-Nicotine has a molar mass of 162.2 g, and levulinic acid molar mass is 116.1 g. In a 1:3 molar ratio, the percentage of nicotine in nicotine levulinate by weight is given by: 162.2 g/(162.2 g+(3×116.1 g))=31.8% (w/w).

### Example 5: Test Formulation 2 (TF2)

A solution of free base nicotine in glycerol comprising 0.40 g (4.00% w/w) of L-nicotine was dissolved in 9.60 g (96.0% w/w) of glycerol and mixed thoroughly.

### Example 6: Heart Rate Study of Nicotine Solutions Via E-Cigarette

Both formulations (TF1 and TF2) were administered in the same fashion by an electronic cigarette to the same human subject: about 0.6 mL of each solution was loaded into “eGo-C” cartridge atomizer (joyetech.com). The atomizer was then attached to an “eVic” e-cigarette (same manufacturer). This model of e-cigarette allows for adjustable voltage, and therefore wattage, through the atomizer. The operating temperature of the e-cigarette is from about 150° C. to about 250° C., or from about 180° C. to about 220° C.

The atomizer in both cases has resistance 2.4 ohms, and the e-cigarette was set to 4.24V, resulting in 7.49 W of power. (P=V\hat{ }2/R)

Heart rate was measured in a 30-second interval for ten minutes from start of puffing. Test participants took 10 puffs over 3 minutes in each case (solid line (2^{nd} highest peak): cigarette, dark dotted line (highest peak): test formulation 1 (TF1-nicotine salt formulation), light dotted line: test formulation 2 (TF2—nicotine formulation). Comparison between cigarette, TF1, and TF2 is shown in FIG. 2.

It is clearly shown in FIG. 2 that the test formulation with nicotine levulinate (TF1) causes a faster rise in heart rate than just nicotine (TF2). Also, TF1 more closely resembles the rate of increase for a cigarette. Other salts were tried and also found to increase heart rate relative to a pure nicotine solution. Thus, other suitable nicotine salts that cause the similar effect may be used in accordance with the practice of the present embodiments and aspects. For example, other keto acids (alpha-keto acids, beta-keto acids, gamma-keto acids, and the like) such as pyruvic acid, oxaloacetic acid, acetoacetic acid, and the like. This experience of increased heart rate comparable to that of a traditional burned cigarette has not been demonstrated or identified in other electronic cigarette devices, nor has it been demonstrated or identified in low temperature tobacco vaporization devices that do not burn the tobacco, even when a nicotine salt was used (a solution of 20% (W/W) or more of nicotine salt) as an additive to the tobacco. Thus the results from this experiment are surprising and unexpected.

In addition, the data appears to correlate well with the previous findings shown in FIG. 2.

As previously noted in the Satisfaction Study, the nicotine salts formulations with acids having vapor pressures between 20-300 mmHg @ 200° C. provide more satisfaction than the rest, with the exception of the nicotine salt formulation made with pyruvic acid, which has a boiling point of 165° C., as noted in FIG. 3. Based on the findings herein, it was anticipated that these nicotine salt formulations having either:
a Vapor Pressure between 20-300 mmHg @ 200° C.,
a Vapor Pressure>20 mmHg @ 200° C.,
a difference between boiling point and melting point of at least 50° C., and a boiling point greater than 160° C., and a melting point less than 160° C.,
a difference between boiling point and melting point of at least 50° C., and a boiling point greater than 160° C., and a melting point less than 160° C.,
a difference between boiling point and melting point of at least 50° C., and a boiling point at most 40° C. less than operating temperature, and a melting point at least 40° C. lower than operating temperature, or
a combination thereof produce one or more of the following effects:

Tₘₐₓ−Time to maximum blood concentration: Based on the results established herein, a user of an e-cigarette comprising the nicotine salt formulation will experience a comparable rate of physical and emotional satisfaction from using a formulation comprising a mixture of nicotine salts prepared with an appropriate acid at least 1.2× to 3× faster than using a formulation comprising a freebase nicotine. As illustrated in FIG. 1: Nicotine from a nicotine salts formulation appears to generate a heartbeat that is nearly 1.2 times that of a normal heart rate for an individual approximately 40 seconds after the commencement of puffing; whereas the nicotine from a nicotine freebase formulation appears to generate a heartbeat that is nearly 1.2 times that of a normal heart rate for an individual approximately 110 seconds after the commencement of puffing; a 2.75× difference in time to achieve a comparable initial satisfaction level.

Again this would not be inconsistent with the data from FIG. 2, where the data illustrated that at approximately 120 seconds (2 minutes), the heart rate of test participants reached a maximum of 105-110 bpm with either a regular cigarette or a nicotine salt formulation (TF1); whereas those same participants heart rates only reached a maximum of approximately 86 bpm at approximately 7 minutes with a nicotine freebase formulation (TF2); also a difference in effect of 1.2 times greater with nicotine salts (and regular cigarettes) versus freebase nicotine.

Further, when considering peak satisfaction levels (achieved at approximately 120 seconds from the initiation of puffing (time=0) and looking at the slope of the line for a normalized heart rate, the approximate slope of those nicotine salt formulations that exceeded the freebase nicotine formulation range between 0.0054 hrₙ/sec and 0.0025 hrₙ/sec. By comparison, the slope of the line for the freebase nicotine formulation is about 0.002. This would suggest that the concentration of available nicotine will be delivered to the user at a rate that is between 1.25 and 2.7 times faster than a freebase formulation.

In another measure of performance; Cₘₐₓ−Maximum blood nicotine concentration; it is anticipated that similar rates of increase will be measured in blood nicotine concentration, as those illustrated above. That is, it was anticipated based on the findings herein, and unexpected based on the art known to date, that there would be comparable Cₘₐₓ between the common cigarette and certain nicotine salt formulations, but with a lower Cₘₐₓ in a freebase nicotine solution.

Similarly, anticipated based on the findings herein, and unexpected based on the art known to date, that certain nicotine salt formulations would have higher rate of nicotine uptake levels in the blood at early time periods. Indeed, Example 8 presents data for multiple salt formulations consistent with these predictions which were made based on the findings and tests noted herein, and unexpected compared to the art available to date.

### Example 7: Heart Rate Study of Nicotine Solutions Via E-Cigarette

Exemplary formulations of nicotine levulinate, nicotine benzoate, nicotine succinate, nicotine salicylate, nicotine malate, nicotine pyruvate, nicotine citrate, nicotine sorbate, nicotine laurate, nicotine freebase, and a control of propylene glycol are prepared as noted in Example 1 and are administered in the same fashion by an electronic cigarette to the same human subject. About 0.5 mL of each solution is loaded into an “eRoll” cartridge atomizer (joyetech.com) to be used in the study. The atomizer is then attached to an “eRoll” e-cigarette (same manufacturer). The operating temperature of the e-cigarette is from about 150° C. to about 250° C., or from about 180° C. to about 220° C.

Heart rate measurements are taken for 6 minutes; from 1 minute before start of puffing, for 3 minutes during puffing, and continuing until 2 minutes after end of puffing. The test participant takes 10 puffs over 3 minutes in each case. The base heart rate is the average heart rate over the first 1 minute before start of puffing. Heart rate after puffing started is averaged over 20-second intervals. Normalized heart rate is defined as the ratio between individual heart rate data point and the base heart rate. Final results are presented as normalized heart rate.

### Example 8: Blood Plasma Testing

Blood plasma testing was conducted on three subjects (n=3). Eight test articles were used in this study: one reference cigarette and seven blends used in an e-cigarette device having an operating temperature of the e-cigarette from about 150° C. to about 250° C., or from about 180° C. to about 220° C. The reference cigarette was Pall Mall (New Zealand). Seven blends were tested in the e-cigarette: 2% free base, 2% benzoate, 4% benzoate, 2% citrate, 2% malate, 2% salicylate, and 2% succinate. Except for 2% succinate (n=1), all other blends have n=3. The seven blends were liquid formulations prepared as described in Example 1.

The concentration of nicotine in each of the formulations was confirmed using UV spectrophotometer (Cary 60, manufactured by Agilent). The sample solutions for UV analysis were made by dissolving 20 mg of each of the formulations in 20 mL 0.3% HCl in water. The sample solutions were then scanned in UV spectrophotometer and the characteristic nicotine peak at 259 nm was used to quantify nicotine in the sample against a standard solution of 19.8 □g/mL nicotine in the same diluent. The standard solution was prepared by first dissolving 19.8 mg nicotine in 10 mL 0.3% HCl in water followed by a 1:100 dilution with 0.3% HCl in water. Nicotine concentrations reported for all formulations were within the range of 95%-105% of the claimed concentrations

All subjects were able to consume 30-55 mg of the liquid formulation of each tested blend using the e-cigarette.

Literature results: C. Bullen et al, Tobacco Control 2010, 19:98-103
Cigarette (5 min adlib, n=9): Tₘₐₓ=14.3 (8.8-19.9), Cₘₐₓ=13.4 (6.5-20.3)
1.4% E-cig (5 min adlib, n=8): Tₘₐₓ=19.6 (4.9-34.2), Cₘₐₓ=1.3 (0.0-2.6)
Nicorette Inhalator (20 mg/20 min, n=10): Tₘₐₓ=32.0 (18.7-45.3), Cₘₐₓ=2.1 (1.0-3.1)

Estimated Cₘₐₓ of 2% nicotine blends:
Cₘₐₓ=Mass consumed*Strength*Bioavailability/(Vol of Distribution*Body Weight)=40 mg*2%*80%/(2.6 L/kg*75kg)=3.3 ng/mL

Estimated Cₘₐₓ of 4% nicotine blends:

Cₘₐₓ=Mass consumed*Strength*Bioavailability/(Vol of Distribution*Body Weight)=40 mg*4%*80%/(2.L/kg*75 kg)=6.6 ng/mL

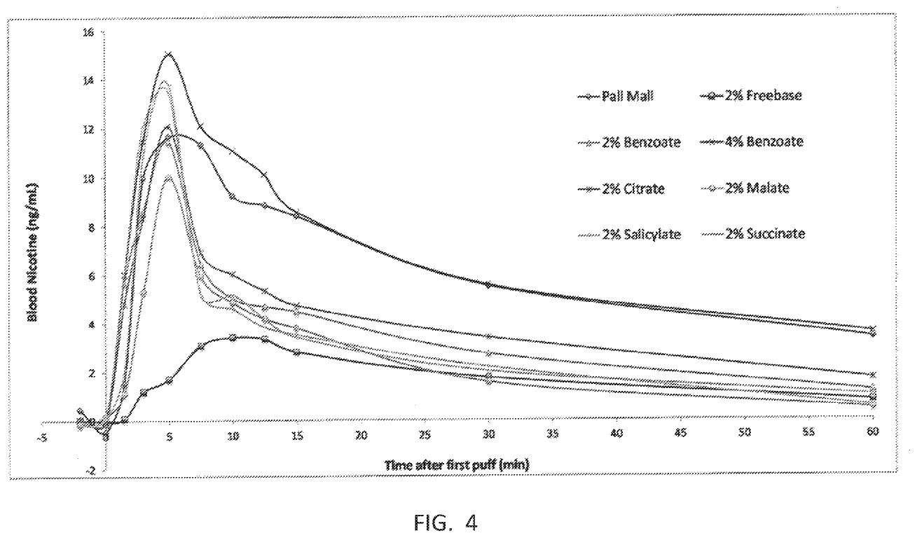

Pharmacokinetic profiles of the blood plasma testing are shown in FIG. 4; showing blood nicotine concentrations (ng/mL) over time after the first puff (inhalation) of the aerosol from the e-cigarette or the smoke of the Pall Mall. Ten puffs were taken at 30 sec intervals starting at time=0 and continuing for 4.5 minutes. For ease of reference and review of FIG. 4, at the 5-minute timepoint, the curves on the graph show from top to bottom (highest average blood nicotine concentration to lowest average blood nicotine concentration) are 4% benzoate, 2% succinate, 2% salicylate, 2% citrate, Pall Mall cigarette, 2% benzoate, 2% malate, and 2% free base blend. Although noted as highest to lowest at this time point, this is not to say that there is a statistically significant difference between any of the salt formulations, or between any of the salt formulations and the Pall Mall cigarette. However, it is possible there may be a statistically significant difference between the Cₘₐₓ of particular salt formulations, and it is also likely based on the data shown in FIG. 4 and in other studies herein that the freebase formulation is statistically different from salt formulations and/or the Pall Mall with respect to Cₘₐₓ, since it appears lower than others tested at several time points. One of skill in the art, upon review of the disclosure herein could properly power a test to determine actual statistically-based differences between one or more formulations and the cigarette, or between the formulations themselves in an e-cigarette. For ease of reference Tables 1 & 2 present the amount of nicotine detected (as an average of all users) for each formulation and the Pall Mall, presented in ng/mL, along with Cₘₐₓ and Tₘₐₓ and AUC. Data from these tables, along with the raw data therefore, was used to generate FIGS. 4, 5, and 6.

TABLE 1

| | | 2% | 2% | 4% |
|---|---|---|---|---|
| Time | Pall Mall | Freebase | Benzoate | Benzoate |
| −2 | 0.46 | 0.03 | 0.09 | 0.05 |
| 0 | −0.46 | −0.03 | −0.09 | −0.05 |
| 1.5 | 1.54 | 0.08 | 5.67 | 6.02 |
| 3 | 9.98 | 1.19 | 8.60 | 11.47 |
| 5 | 11.65 | 1.70 | 11.44 | 15.06 |
| 7.5 | 11.34 | 3.09 | 6.43 | 12.12 |
| 10 | 9.24 | 3.42 | 5.03 | 11.08 |
| 12.5 | 8.85 | 3.35 | 4.68 | 10.10 |
| 15 | 8.40 | 2.81 | 4.47 | 8.57 |
| 30 | 5.51 | 1.74 | 2.72 | 5.56 |
| 60 | 3.39 | 0.79 | 1.19 | 3.60 |
| Tₘₐₓ (min) | 5.17 | 10.00 | 6.67 | 5.83 |
| Cₘₐₓ (ng/mL) | 11.65 | 3.42 | 11.44 | 15.06 |
| AUC | 367.5 | 106.2 | 207.8 | 400.2 |
| (ng*min/mL) | | | | |

TABLE 2

| | 2% | 2% | 2% | 2% |
|---|---|---|---|---|
| Time | Citrate | Malate | Salicylate | Succinate |
| −2 | 0.06 | −0.17 | −0.19 | −0.06 |
| 0 | −0.06 | 0.17 | 0.19 | 0.06 |
| 1.5 | 4.80 | 1.09 | 6.14 | 2.10 |
| 3 | 8.33 | 5.30 | 12.04 | 10.81 |
| 5 | 12.09 | 10.02 | 13.46 | 13.81 |
| 7.5 | 6.93 | 5.93 | 5.21 | 5.15 |
| 10 | 6.01 | 4.85 | 4.60 | 5.18 |
| 12.5 | 5.34 | 4.17 | 3.83 | 4.17 |
| 15 | 4.72 | 3.79 | 3.52 | 3.41 |
| 30 | 3.40 | 1.56 | 2.19 | 2.01 |
| 60 | 1.70 | 0.46 | 0.55 | 1.00 |
| Tₘₐₓ (min) | 5.83 | 5.00 | 4.33 | 5.00 |
| Cₘₐₓ (ng/mL) | 12.09 | 10.02 | 13.46 | 13.81 |
| AUC | 238.0 | 146.1 | 182.9 | 179.5 |
| (ng*min/mL) | | | | |

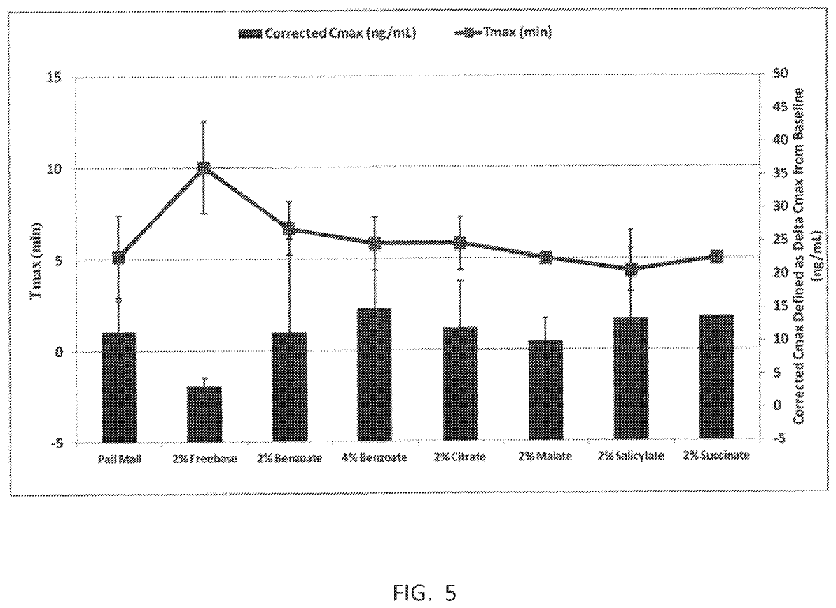

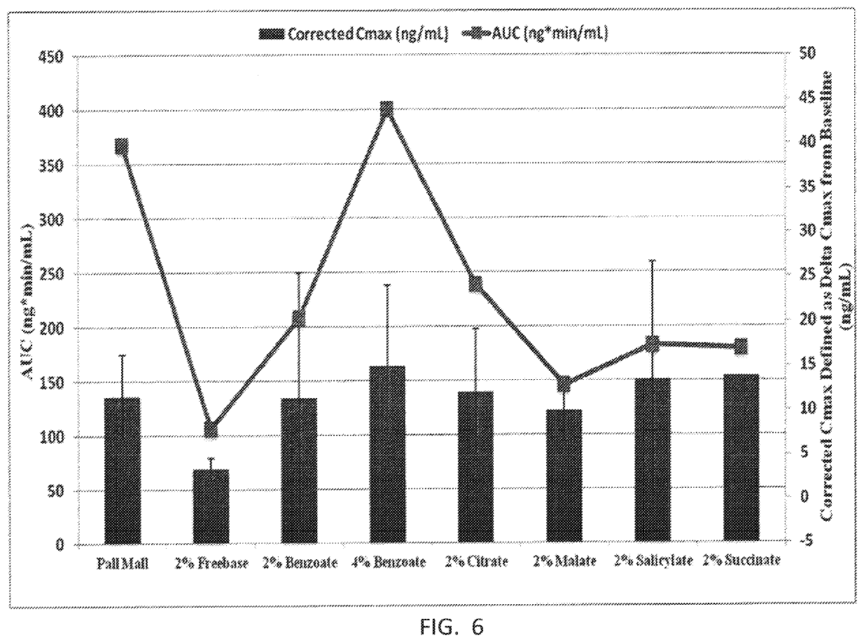

Comparison of Tₘₐₓ and Cₘₐₓ of the seven blends and reference cigarette are shown in FIG. 5. Comparison of Cₘₐₓ and AUC of the seven blends and reference cigarette are shown in FIG. 6. Due to the time limit of the wash-period, baseline blood nicotine concentration (at t=−2 and t=0 min) was higher for samples consumed at a later time on the test day. The data in FIGS. 4-6 show corrected blood nicotine concentration values (i.e. apparent blood nicotine concentration at each time point minus baseline nicotine concentration of the same sample).

Rates of nicotine uptake in the blood of the users of each sample within the first 90 seconds are shown in Table 3.

TABLE 3

| Sample | Rate of nicotine uptake (ng/mL/min) |
|---|---|
| 2% Salicylate | 4.09 |
| 2% Benzoate | 3.78 |
| 2% Citrate | 3.20 |
| 2% Succinate | 1.40 |
| Pall Mall (reference) | 1.03 |
| 2% Malate | 0.73 |
| 2% Freebase | 0.05 |
| 4% Benzoate | 4.01 |

Although the Tₘₐₓ and Cₘₐₓ values are comparable between the tested blends and the reference cigarette (with the exception of the 2% free base blend), the rates of nicotine absorption within the first 90 seconds differed among the test articles. In particular, four blends (2% salicylate, 2% benzoate, 4% benzoate, and 2% citrate) showed markedly higher rates of absorption within the first 90 seconds compared to the other blends and with the reference cigarette. These four blends contain salts (salicylate, benzoate, and citrate) which performed well in the Satisfaction Study of Example 3. Moreover, 2% benzoate and 4% benzoate had comparable rates of absorption, suggesting that a lower concentration of nicotinic salt may not adversely impact the rate of absorption.

### Example 9: Blood Plasma Testing

Blood plasma testing is conducted on 24 subjects (n=24). Eight test articles are used in this study: one reference cigarette and seven blends delivered to a user in an e-cigarette as an aerosol. The operating temperature of the e-cigarette is from about 150° C. to about 250° C., or from about 180° C. to about 220° C. The reference cigarette is Pall Mall (New Zealand). Seven blends are tested: 2% free base, 2% benzoate, 4% benzoate, 2% citrate, 2% malate, 2% salicylate, and 2% succinate. The seven blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0) Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 10: Blood Plasma Testing

Blood plasma testing is conducted on twenty-four subjects (n=24). Eleven test articles are used in this study: one reference cigarette and ten blends delivered to a user in an e-cigarette as an aerosol. The reference cigarette is Pall Mall (New Zealand). The operating temperature of the e-cigarette is from about 150° C. to about 250° C., or from about 180° C. to about 220° C. Ten blends are tested: 2% free base, 2% benzoate, 2% sorbate, 2% pyruvate, 2% laurate, 2% levulinate, 2% citrate, 2% malate, 2% salicylate, and 2% succinate. The ten blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 11: Blood Plasma Testing

Blood plasma testing is conducted on twenty-four subjects (n=24). Twenty-one test articles are used in this study: one reference cigarette and twenty blends delivered to a user in an e-cigarette as an aerosol. The reference cigarette is Pall Mall (New Zealand). The operating temperature of the e-cigarette is from about 150° C. to about 250° C., or from about 180° C. to about 220° C. Twenty blends are tested: 2% free base, 4% free base, 2% benzoate, 4% benzoate, 2% sorbate, 4% sorbate, 2% pyruvate, 4% pyruvate, 2% laurate, 4% laurate, 2% levulinate, 4% levulinate, 2% citrate, 4% citrate, 2% malate, 4% malate, 2% salicylate, 4% salicylate, 2% succinate, and 4% succinate. The twenty blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 12: Blood Plasma Testing

Blood plasma testing is conducted on twenty-four subjects (n=24). Twenty-one test articles are used in this study: one reference cigarette and twenty blends delivered to a user in an e-cigarette as an aerosol. The reference cigarette is Pall Mall (New Zealand). The operating temperature of the e-cigarette is from about 150° C. to about 250° C., or from about 180° C. to about 220° C. Twenty blends are tested: 2% free base, 1% free base, 2% benzoate, 1% benzoate, 2% sorbate, 1% sorbate, 2% pyruvate, 1% pyruvate, 2% laurate, 1% laurate, 2% levulinate, 1% levulinate, 2% citrate, 1% citrate, 2% malate, 1% malate, 2% salicylate, 1% salicylate, 2% succinate, and 1% succinate. The twenty blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 13. Preparation of Liquid Nicotine: Oxalic Acid Formulation

A liquid nicotine formulation comprising a 1:1 molar ratio of nicotine:oxalic acid (1:2 nicotine:oxalic acid functional groups) is prepared in accordance with Example 1 by adding the required amount of Oxalic Acid (˜2.7-2.8% of total formulation weight) into a clean empty vial. The required amount of Nicotine (5% of total formulation weight) is added to the same vial. A required amount of pre-mixed PG:VG 30:70 (92.2% of total formulation weight) is added to the same vial. The vial cap is closed and placed in a water bath at ˜60 C.° and vortexed until all oxalic acid is dissolved based on visual inspection.

Alternatively, liquid nicotine formulation comprising a 1:1 molar ratio of nicotine:oxalic acid (1:2 nicotine:oxalic acid functional groups) is prepared in accordance with Example 1 by adding the required amount of Oxalic Acid (−2.7-2.8% of total formulation weight) into a clean empty vial. The required amount of PG (30%×92.2%=27.7% of total formulation weight) is added to the vial to form an Oxalic Acid-PG solution. The vial cap is closed and placed in a water bath at ˜60 C° and vortexed until all oxalic acid is dissolved based on visual inspection. Following cooling of the vial at room temperature, Nicotine and required amount of VG (70%×92.2%=64.5% of total formulation weight) is added to the Oxalic-PG solution. The mixture is slightly heated until viscosity is reduced and then vortexed until well mixed based on visual inspection.

### Example 14. Harshness Study of Liquid Nicotine: Oxalic Acid Formulation Via E-Cigarette

A 3% w/w nicotine formulation as described in Example 1 and a liquid nicotine formulation as described in Example 13 are used to conduct a harshness study in a single test participant. The nicotine formulations have the same or similar masking flavors and are blind coded. The test participant, an e-cigarette and/or traditional cigarette user, is required to have no nicotine intake for at least 12 hours before the test. Each test participant takes 10 puffs using an e-cigarette (same as used in Example 2) over 3 minutes in each case. The participants are asked to rate the level of physical and/or emotional satisfaction he or she felt on a scale of 0-10, with 0 being no physical or emotional satisfaction.

Although various illustrative embodiments are described above, any of a number of changes may be made to various embodiments without departing from the scope of the embodiments and aspects and embodiments described herein. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the aspects and embodiments described herein.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Preferred embodiments of the present disclosure have been shown and described herein, it will be apparent to those skilled in the art that such embodiments are provided by way of example only. Numerous variations, changes, and substitutions will now occur to those skilled in the art without departing from the aspects and embodiments described herein. It should be understood that various alternatives to the embodiments of the embodiments described herein can be employed and practiced.

What is claimed is:

1. A method of delivering protonated nicotine to a user of an electronic cigarette, the method comprising:
(a) operating an electronic cigarette comprising a liquid nicotine formulation, said liquid nicotine comprising nicotine, oxalic acid and a biologically acceptable liquid carrier;
(b) heating said liquid nicotine formulation to an operating temperature, wherein said heating provides an inhalable aerosol comprising an effective amount of protonated nicotine; and
(c) inhaling said inhalable aerosol.

2. The method of claim 1, wherein said oxalic acid and said nicotine form a nicotine salt.

3. The method of claim 1 or 2, wherein the molar ratio of nicotine to oxalic acid functional groups is about 1:2 or greater.

4. The method of any of claims 1 to 3, wherein the molar ratio of nicotine to oxalic acid is from about 1:0.5 to about 1:1.5.

5. The method of any one of claims 1 to 4, wherein the molar ratio of nicotine to oxalic acid is about 1:1.

6. The method of any one of claims 1 to 5, wherein the moles of oxalic acid functional groups is equal to or greater than the moles of nicotine.

7. The method of any one of claims 1 to 6 wherein the moles of oxalic acid functional groups is equal to or greater than the moles of nicotine.

8. The method of any one of claims 1 to 7, wherein the moles of oxalic acid functional groups is greater than the moles of nicotine.

9. The method of any one of claims 1 to 8, wherein the moles of oxalic acid functional groups is from about 1.1 times greater to about 3.0 times greater than the moles of nicotine.

10. The method of any one of claims 1 to 9, wherein the moles of oxalic acid functional groups is from about 1.5 times greater to about 2.2 times greater than the moles of nicotine.

11. The method of any one of claims 1 to 10, wherein the liquid nicotine formulation comprises about 4% to about 5% nicotine (w/w) and about 2.5% to about 3.5% oxalic acid (w/w).

12. The method of any one of claims 1 to 11, wherein the liquid nicotine formulation comprises about 5% nicotine (w/w) and about 2.8% (w/w) oxalic acid.

13. The method of any one of claims 1 to 12, wherein the nicotine salt is in an amount that forms about 1% to about 20% nicotine in the inhalable aerosol.

14. The method of any one of claims 1 to 13, wherein the liquid nicotine formulation has a nicotine concentration of from about 0.5% (w/w) to about 20% (w/w).

15. The method of any one of claims 1 to 14, wherein the operating temperature is from about150° C. to about 250° C.

16. The method of any one of claims 1 to 15, wherein the operating temperature is from about 180° C. to about 220° C.

17. The method of any one of claims 1 to 16, wherein the operating temperature is 200° C.

18. The method of any one of claims 1 to 17, wherein the aerosol comprises a condensate of the nicotine salt.

19. The method of any one of claims 1 to 18, wherein said nicotine salt is nicotine oxalate.

20. The method any one of claims 1 to 19, wherein the aerosol comprises condensate in particle sizes from about 0.1 microns to about 5 microns.

21. The method any one of claims 1 to 20, wherein the aerosol comprises condensate in particle sizes from about from about 0.1 microns to about 1 or 2 microns.

22. The method any one of claims 1 to 21, wherein the aerosol comprises condensate in particle sizes from about from about 0.1 microns to about 0.7 microns.

23. The method any one of claims 1 to 22, wherein the aerosol comprises condensate in particle sizes from about from about 0.3 microns to about 0.4 microns.

24. The method of any one of claims 1 to 23, wherein the biologically acceptable liquid carrier comprises glycerol, propylene glycol, trimethylene glycol, water, ethanol or combinations thereof.

25. The method of any one of claims 1 to 24, wherein the biologically acceptable liquid carrier comprises propylene glycol and vegetable glycerin.

26. The method of any one of claims 1 to 25, wherein the biologically acceptable liquid carrier comprises about 20% to about 50% of propylene glycol and about 80% to about 50% of vegetable glycerin.

27. The method of any one of claims 1 to 26, wherein the biologically acceptable liquid carrier comprises about 30% propylene glycol and about 70% vegetable glycerin.

28. The method of any one of claims 1 to 27, wherein the nicotine salt is in an amount that forms about 0.5% to about 20% nicotine in the inhalable aerosol or about 1% to about 20% nicotine in the inhalable aerosol.

29. The method of any one of claims 1 to 28, wherein the formulation further comprises one or more additional nicotine salts in a biologically acceptable biologically acceptable liquid carrier suitable for generating the inhalable aerosol upon heating.

30. The method of claim 29, wherein an additional acid is used to form the additional nicotine salt.

31. The method of claim 30, wherein the additional acid is selected from the group consisting of salicylic acid, sorbic acid, benzoic acid, pyruvic acid, lauric acid, and levulinic acid.

32. The method of any one of claims 1 to 31, wherein the formulation further comprises a flavorant.

33. The method of any one of claims 1 to 32, wherein said effective amount results in said user experiencing less respiratory tract harshness relative to a control liquid nicotine formulation comprising an equivalent amount of an acid other than oxalic acid.

34. A liquid nicotine formulation comprising nicotine, oxalic acid and a biologically acceptable liquid biologically compatible carrier, wherein upon heating said liquid nicotine formulation an inhalable aerosol is formed comprising an effective amount of protonated nicotine.

35. The liquid nicotine formulation of claim 34, wherein said liquid nicotine formulation is in a cartridge.

36. The liquid nicotine formulation of claim 35, wherein said cartridge is in an electronic cigarette.

37. The liquid nicotine formulation of claim 34, wherein said oxalic acid and said nicotine form a nicotine salt.

38. The liquid nicotine formulation of claim 34 or 37, wherein the molar ratio of nicotine to oxalic acid functional groups is about 1:2 or greater.

39. The liquid nicotine formulation of any of claims 34 to 38, wherein the molar ratio of nicotine to oxalic acid is from about 1:0.5 to about 1:1.5.

40. The liquid nicotine formulation of any one of claims 34 to 39, wherein the molar ratio of nicotine to oxalic acid is about 1:1.

41. The liquid nicotine formulation of any one of claims 34 to 40, wherein the moles of oxalic acid functional groups is equal to or greater than the moles of nicotine.

42. The liquid nicotine formulation of any one of claims 34 to 41, wherein the moles of oxalic acid functional groups is greater than the moles of nicotine.

43. The liquid nicotine formulation of any one of claims 34 to 42, wherein the moles of oxalic acid functional groups is equal to or greater than the moles of nicotine.

44. The liquid nicotine formulation of any one of claims 34 to 43, wherein the moles of oxalic acid functional groups is from about 1.1 times greater to about 3.0 times greater than the moles of nicotine.

45. The liquid nicotine formulation of any one of claims 34 to 44, wherein the moles of oxalic acid functional groups is from about 1.5 times greater to about 2.2 times greater than the moles of nicotine.

46. The liquid nicotine formulation of any one of claims 34 to 45, wherein the liquid nicotine formulation comprises about 4% to about 5% nicotine (w/w) and about 2.5% to about 3.5% oxalic acid (w/w).

47. The liquid nicotine formulation of any one of claims 34 to 46, wherein the liquid nicotine formulation comprises about 5% nicotine (w/w) and about 2.8% (w/w) oxalic acid.

48. The liquid nicotine formulation of any one of claims 34 to 47, wherein the nicotine salt is in an amount that forms about 1% to about 20% nicotine in the inhalable aerosol.

49. The liquid nicotine formulation of any one of claims 34 to 48, wherein the liquid nicotine formulation has a nicotine concentration of from about 0.5% (w/w) to about 20% (w/w).

50. The liquid nicotine formulation of any one of claims 34 to 49, wherein the operating temperature is from about150° C. to about 250° C.

51. The liquid nicotine formulation of any one of claims 34 to 50, wherein the operating temperature is from about 180° C. to about 220° C.

52. The liquid nicotine formulation of any one of claims 34 to 51, wherein the operating temperature is 200° C.

53. The liquid nicotine formulation of any one of claims 34 to 52, wherein the aerosol comprises a condensate of the nicotine salt.

54. The liquid nicotine formulation of any one of claims 34 to 53, wherein said nicotine salt is nicotine oxalate.

55. The liquid nicotine formulation any one of claims 34 to 54, wherein the aerosol comprises condensate in particle sizes from about 0.1 microns to about 5 microns.

56. The liquid nicotine formulation any one of claims 34 to 55, wherein the aerosol comprises condensate in particle sizes from about from about 0.1 microns to about 1 or 2 microns.

57. The liquid nicotine formulation any one of claims 34 to 56, wherein the aerosol comprises condensate in particle sizes from about from about 0.1 microns to about 0.7 microns.

58. The liquid nicotine formulation any one of claims 34 to 57, wherein the aerosol comprises condensate in particle sizes from about from about 0.3 microns to about 0.4 microns.

59. The liquid nicotine formulation of any one of claims 34 to 58, wherein the biologically acceptable liquid carrier comprises glycerol, propylene glycol, trimethylene glycol, water, ethanol or combinations thereof.

60. The liquid nicotine formulation of any one of claims 34 to 59, wherein the biologically acceptable liquid carrier comprises propylene glycol and vegetable glycerin.

61. The liquid nicotine formulation of any one of claims 34 to 60, wherein the biologically acceptable liquid carrier comprises about 20% to about 50% of propylene glycol and about 80% to about 50% of vegetable glycerin.

62. The liquid nicotine formulation of any one of claims 34 to 61, wherein the biologically acceptable liquid carrier comprises about 30% propylene glycol and about 70% vegetable glycerin.

63. The liquid nicotine formulation of any one of claims 34 to 62, wherein the nicotine salt is in an amount that forms about 0.5% to about 20% nicotine in the inhalable aerosol or about 1% to about 20% nicotine in the inhalable aerosol.

64. The liquid nicotine formulation of any one of claims 34 to 63, wherein the formulation further comprises one or more additional nicotine salts in a biologically acceptable liquid carrier suitable for generating the inhalable aerosol upon heating.
